SEQUENCE LISTING (20 sequences)

SEQ ID NO: 1 - DNA, 1317 bp, Artificial Sequence
  Description of Artificial Sequence Recombinant DNA
atggttcaatggaagagactctgccagctgcattacttgtgggctctgggctgctatatgctgctggccactgtggctctgaaactttctttcaggttgaagtgtgactctgaccacttgggtctggagtccagggaatctcaaagccagtactgtaggaatatcttgtataatttcctgaaacttccagcaaagaggtctatcaactgttcaggggtcacccgaggggaccaagaggcagtgcttcaggctattctgaataacctggaggtcaagaagaagcgagagcctttcacagacacccactacctctccctcaccagagactgtgagcacttcaaggctgaaaggaagttcatacagttcccactgagcaaagaagaggtggagttccctattgcatactctatggtgattcatgagaagattgaaaactttgaaaggctactgcgagctgtgtatgcccctcagaacatatactgtgtccatgtggatgagaagtccccagaaactttcaaagaggcggtcaaagcaattatttcttgcttcccaaatgtcttcatagccagtaagctggttcgggtggtttatgcctcctggtccagggtgcaagctgacctcaactgcatggaagacttgctccagagctcagtgccgtggaaatacttcctgaatacatgtgggacggactttcctataaagagcaatgcagagatggtccaggctctcaagatgttgaatgggaggaatagcatggagtcagaggtacctcctaagcacaaagaaacccgctggaaatatcactttgaggtagtgagagacacattacacctaaccaacaagaagaaggatcctcccccttataatttaactatgtttacagggaatgcgtacattgtggcttcccgagatttcgtccaacatgttttgaagaaccctaaatcccaacaactgattgaatgggtaaaagacacttatagcccagatgaacacctctgggccacccttcagcgtgcacggtggatgcctggctctgttcccaaccaccccaagtacgacatctcagacatgacttctattgccaggctggtcaagtggcagggtcatgagggagacatcgataagggtgctccttatgctccctgctctggaatccaccagcgggctatctgcgtttatggggctggggacttgaattggatgcttcaaaaccatcacctgttggccaacaagtttgacccaaaggtagatgataatgctcttcagtgcttagaagaatacctacgttataaggccatctatgggactgaactttga

SEQ ID NO: 2 - protein, 438 aa, Artificial Sequence
  Description of Artificial Sequence Recombinant amino acid
MVQWKRLCQLHYLWALGCYMLLATVALKLSFRLKCDSDHLGLESRESQSQYCRNILYNFLKLPAKRSINCSGVTRGDQEAVLQAILNNLEVKKKREPFTDTHYLSLTRDCEHFKAERKFIQFPLSKEEVEFPIAYSMVIHEKIENFERLLRAVYAPQNIYCVHVDEKSPETFKEAVKAIISCFPNVFIASKLVRVVYASWSRVQADLNCMEDLLQSSVPWKYFLNTCGTDFPIKSNAEMVQALKMLNGRNSMESEVPPKHKETRWKYHFEVVRDTLHLTNKKKDPPPYNLTMFTGNAYIVASRDFVQHVLKNPKSQQLIEWVKDTYSPDEHLWATLQRARWMPGSVPNHPKYDISDMTSIARLVKWQGHEGDIDKGAPYAPCSGIHQRAICVYGAGDLNWMLQNHHLLANKFDPKVDDNALQCLEEYLRYKAIYGTEL

SEQ ID NO: 3 - DNA, 2108 bp, Artificial Sequence
  Description of Artificial Sequence Recombinant DNA
atctgcttcctggttctatattaaagaggagcctgaaactgttccttggacatcttatgaatgtcagaaaataccttttggagggttagaagatcaggggacatggttgttcacatttgctgccacggaacaccgccagtcttcacttggaaacagaatcacgccttgtgaagagatcatccctaagcaggagagaagctactaaaggattgtgtcctcctccaccttccctgtgctcggtctccacctgtctcccattctgtgacgatggttcaatggaagagactctgccagctgcattacttgtgggctctgggctgctatatgctgctggccactgtggctctgaaactttctttcaggttgaagtgtgactctgaccacttgggtctggagtccagggaatctcaaagccagtactgtaggaatatcttgtataatttcctgaaacttccagcaaagaggtctatcaactgttcaggggtcacccgaggggaccaagaggcagtgcttcaggctattctgaataacctggaggtcaagaagaagcgagagcctttcacagacacccactacctctccctcaccagagactgtgagcacttcaaggctgaaaggaagttcatacagttcccactgagcaaagaagaggtggagttccctattgcatactctatggtgattcatgagaagattgaaaactttgaaaggctactgcgagctgtgtatgcccctcagaacatatactgtgtccatgtggatgagaagtccccagaaactttcaaagaggcggtcaaagcaattatttcttgcttcccaaatgtcttcatagccagtaagctggttcgggtggtttatgcctcctggtccagggtgcaagctgacctcaactgcatggaagacttgctccagagctcagtgccgtggaaatacttcctgaatacatgtgggacggactttcctataaagagcaatgcagagatggtccaggctctcaagatgttgaatgggaggaatagcatggagtcagaggtacctcctaagcacaaagaaacccgctggaaatatcactttgaggtagtgagagacacattacacctaaccaacaagaagaaggatcctcccccttataatttaactatgtttacagggaatgcgtacattgtggcttcccgagatttcgtccaacatgttttgaagaaccctaaatcccaacaactgattgaatgggtaaaagacacttatagcccagatgaacacctctgggccacccttcagcgtgcacggtggatgcctggctctgttcccaaccaccccaagtacgacatctcagacatgacttctattgccaggctggtcaagtggcagggtcatgagggagacatcgataagggtgctccttatgctccctgctctggaatccaccagcgggctatctgcgtttatggggctggggacttgaattggatgcttcaaaaccatcacctgttggccaacaagtttgacccaaaggtagatgataatgctcttcagtgcttagaagaatacctacgttataaggccatctatgggactgaactttgagacacactatgagagcgttgctacctgtggggcaagagcatgtacaaacatgctcagaacttgctgggacagtgtgggtgggagaccagggctttgcaattcgtggcatcctttaggataagagggctgctattagattgtgggtaagtagatcttttgccttgcaaattgctgcctgggtgaatgctgcttgttctctcacccctaaccctagtagttcctccactaactttctcactaagtgagaatgagaactgctgtgatagggagagtgaaggagggatatgtggtagagcacttgatttcagttgaatgcctgctggtagcttttccattctgtggagctgccgttcctaataattccaggtttggtagcgtggaggagaactttgatggaaagagaaccttcccttctgtactgttaacttaaaaataaatagctcctgattcaaagtaaaaaaaaaaaaaaaaaaaaaaaaaaaaaaaaaaaaaaaaaaaaaaaaaaaaaaagggagctcgaattc

SEQ ID NO: 4 - protein, 663 aa, Artificial Sequence
  Description of Artificial Sequence Recombinant amino acid
ICFLVLYRGANCSLDILMSENTFWRVRRSGDMVVHICCHGTPPVFTWKQNHALRDHPAGEKLLKDCVLLHLPCARSPPVSHSVTMVQWKRLCQLHYLWALGCYMLLATVALKLSFRLKCDSDHLGLESRESQSQYCRNILYNFLKLPAKRSINCSGVTRGDQEAVLQAILNNLEVKKKREPFTDTHYLSLTRDCEHFKAERKFIQFPLSKEEVEFPIAYSMVIHEKIENFERLLRAVYAPQNIYCVHVDEKSPETFKEAVKAIISCFPNVFIASKLVRVVYASWSRVQADLNCMEDLLQSSVPWKYFLNTCGTDFPIKSNAEMVQALKMLNGRNSMESEVPPKHKETRWKYHFEVVRDTLHLTNKKKDPPPYNLTMFTGNAYIVASRDFVQHVLKNPKSQQLIEWVKDTYSPDEHLWATLQRARWMPGSVPNHPKYDISDMTSIARLVKWQGHEGDIDKGAPYAPCSGIHQRAICVYGAGDLNWMLQNHHLLANKFDPKVDDNALQCLEEYLRYKAIYGTELDTLERCYLWGKSMYKHAQNLLGQCGWETRALQFVASFRIRGLLLDCGVDLLPCKLLPGMLLVLSPLTLVVPPLTFSLSENENCCDRESEGGICGRALDFSMPAGSFSILWSCRSFQVWRGGELWKENLPFCTVNLKINSSF

SEQ ID NO: 5 - DNA, 524 bp, Artificial Sequence
  Description of Artificial Sequence Recombinant DNA
gacacactatgagagcgttgctacctgtggggcaagagcatgtacaaacatgctcagaacttgctgggacagtgtgggtgggagaccagggctttgcaattcgtggcatcctttaggataagagggctgctattagattgtgggtaagtagatcttttgccttgcaaattgctgcctgggtgaatgctgcttgttctctcacccctaaccctagtagttcctccactaactttctcactaagtgagaatgagaactgctgtgatagggagagtgaaggagggatatgtggtagagcacttgatttcagttgaatgcctgctggtagcttttccattctgtggagctgccgttcctaataattccaggtttggtagcgtggaggagaactttgatggaaagagaaccttcccttctgtactgttaacttaaaaataaatagctcctgattcaaagtaaaaaaaaaaaaaaaaaaaaaaaaaaaaaaaaaaaaaaaaaaaaaaaaaaaaaaagggagctcgaattc

SEQ ID NO: 6 - DNA, 267 bp, Artificial Sequence
  Description of Artificial Sequence Recombinant DNA
atctgcttcctggttctatattaaagaggagcctgaaactgttccttggacatcttatgaatgtcagaaaataccttttggagggttagaagatcaggggacatggttgttcacatttgctgccacggaacaccgccagtcttcacttggaaacagaatcacgccttgtgaagagatcatccctaagcaggagagaagctactaaaggattgtgtcctcctccaccttccctgtgctcggtctccacctgtctcccattctgtgacg

SEQ ID NO: 7 - protein, 9 aa, Artificial Sequence
  Description of Artificial Sequence Recombinant amino acid
MVQWKRLCQ

SEQ ID NO: 8 - protein, 23 aa, Artificial Sequence
  Description of Artificial Sequence Recombinant amino acid
LHYLWALGCYMLLATVALKLSFR

SEQ ID NO: 9 - protein, 406 aa, Artificial Sequence
  Description of Artificial Sequence Recombinant amino acid
LKCDSDHLGLESRESQSQYCRNILYNFLKLPAKRSINCSGVTRGDQEAVLQAILNNLEVKKKREPFTDTHYLSLTRDCEHFKAERKFIQFPLSKEEVEFPIAYSMVIHEKIENFERLLRAVYAPQNIYCVHVDEKSPETFKEAVKAIISCFPNVFIASKLVRVVYASWSRVQADLNCMEDLLQSSVPWKYFLNTCGTDFPIKSNAEMVQALKMLNGRNSMESEVPPKHKETRWKYHFEVVRDTLHLTNKKKDPPPYNLTMFTGNAYIVASRDFVQHVLKNPKSQQLIEWVKDTYSPDEHLWATLQRARWMPGSVPNHPKYDISDMTSIARLVKWQGHEGDIDKGAPYAPCSGIHQRAICVYGAGDLNWMLQNHHLLANKFDPKVDDNALQCLEEYLRYKAIYGTEL

SEQ ID NO: 10 - DNA, 27 bp, Artificial Sequence
  Description of Artificial Sequence Recombinant DNA
atggttcaatggaagagactctgccag

SEQ ID NO: 11 - DNA, 69 bp, Artificial Sequence
  Description of Artificial Sequence Recombinant DNA
ctgcattacttgtgggctctgggctgctatatgctgctggccactgtggctctgaaactttctttcagg

SEQ ID NO: 12 - DNA, 1221 bp, Artificial Sequence
  Description of Artificial Sequence Recombinant DNA
ttgaagtgtgactctgaccacttgggtctggagtccagggaatctcaaagccagtactgtaggaatatcttgtataatttcctgaaacttccagcaaagaggtctatcaactgttcaggggtcacccgaggggaccaagaggcagtgcttcaggctattctgaataacctggaggtcaagaagaagcgagagcctttcacagacacccactacctctccctcaccagagactgtgagcacttcaaggctgaaaggaagttcatacagttcccactgagcaaagaagaggtggagttccctattgcatactctatggtgattcatgagaagattgaaaactttgaaaggctactgcgagctgtgtatgcccctcagaacatatactgtgtccatgtggatgagaagtccccagaaactttcaaagaggcggtcaaagcaattatttcttgcttcccaaatgtcttcatagccagtaagctggttcgggtggtttatgcctcctggtccagggtgcaagctgacctcaactgcatggaagacttgctccagagctcagtgccgtggaaatacttcctgaatacatgtgggacggactttcctataaagagcaatgcagagatggtccaggctctcaagatgttgaatgggaggaatagcatggagtcagaggtacctcctaagcacaaagaaacccgctggaaatatcactttgaggtagtgagagacacattacacctaaccaacaagaagaaggatcctcccccttataatttaactatgtttacagggaatgcgtacattgtggcttcccgagatttcgtccaacatgttttgaagaaccctaaatcccaacaactgattgaatgggtaaaagacacttatagcccagatgaacacctctgggccacccttcagcgtgcacggtggatgcctggctctgttcccaaccaccccaagtacgacatctcagacatgacttctattgccaggctggtcaagtggcagggtcatgagggagacatcgataagggtgctccttatgctccctgctctggaatccaccagcgggctatctgcgtttatggggctggggacttgaattggatgcttcaaaaccatcacctgttggccaacaagtttgacccaaaggtagatgataatgctcttcagtgcttagaagaatacctacgttataaggccatctatgggactgaactttga

SEQ ID NO: 13 - DNA, 2109 bp, Homo sapiens
gtgaagtgctcagaatggggcaggatgtcacctggaatcagcactaagtgattcagactttccttacttttaaatgtgctgctcttcatttcaagatgccgttgcagctctgataaatgcaaactgacaaccttcaaggccacgacggagggaaaatcattggtgcttggagcatagaagactgcccttcacaaaggaaatccctgattattgtttgaaatgctgaggacgttgctgcgaaggagacttttttcttatcccaccaaatactactttatggttcttgttttatccctaatcaccttctccgttttaaggattcatcaaaagcctgaatttgtaagtgtcagacacttggagcttgctggggagaatcctagtagtgatattaattgcaccaaagttttacagggtgatgtaaatgaaatccaaaaggtaaagcttgagatcctaacagtgaaatttaaaaagcgccctcggtggacacctgacgactatataaacatgaccagtgactgttcttctttcatcaagagacgcaaatatattgtagaaccccttagtaaagaagaggcggagtttccaatagcatattctatagtggttcatcacaagattgaaatgcttgacaggctgctgagggccatctatatgcctcagaatttctattgcgttcatgtggacacaaaatccgaggattcctatttagctgcagtgatgggcatcgcttcctgttttagtaatgtctttgtggccagccgattggagagtgtggtttatgcatcgtggagccgggttcaggctgacctcaactgcatgaaggatctctatgcaatgagtgcaaactggaagtacttgataaatctttgtggtatggattttcccattaaaaccaacctagaaattgtcaggaagctcaagttgttaatgggagaaaacaacctggaaacggagaggatgccatcccataaagaagaaaggtggaagaagcggtatgaggtcgttaatggaaagctgacaaacacagggactgtcaaaatgcttcctccactcgaaacacctctcttttctggcagtgcctacttcgtggtcagtagggagtatgtggggtatgtactacagaatgaaaaaatccaaaagttgatggagtgggcacaagacacatacagccctgatgagtatctctgggccaccatccaaaggattcctgaagtcccgggctcactccctgccagccataagtatgatctatctgacatgcaagcagttgccaggtttgtcaagtggcagtactttgagggtgatgtttccaagggtgctccctacccgccctgcgatggagtccatgtgcgctcagtgtgcattttcggagctggtgactgaactggatgctgcgcaaacaccacttgtttgccaataagtttgacgtggatgttgacctctttgccatccagtgtttggatgagcatttgagacacaaagctttggagacattaaaacactgaccattacgggcaattttatgaacaagaagaaggatacacaaaacgtaccttatctgtttccccttccttgtcagcgtcgggaagatggtatgaagtcctctttggggcagggactctagtagatcttcttgtcagagaagctgcatggtttctgcagagcacagttagctagaaaggtgatagcattaaatgttcatctagagttaatagtgggaggagtaaaggtagccttgaggccagagcaggtagcaaggcattgtggaaagaggggaccagggtggctggggaagaggccgatgcataaagtcagcctgttccaagtgctcagggacttagcaaaatgagaagatgtgacctgtgccaaaactattttgagaattttaaatgtgaccatttttctggtatgccaataaaddcttacagcaacaaataatcaaagatacaattaatctgatattatatttgttgaaatagaaatttgattgtactataaatgatttttgtaaataatttatattctgctctaatactgtactgtgtagtgtgtctccgtatgtcatctcagggagcttaaaatgggcttgatttaacattgaaaaaaaat

SEQ ID NO: 14 - protein, 428 aa, Homo sapiens [sequence omitted]

SEQ ID NO: 15 - DNA, 22 bp, Artificial Sequence
  Description of Artificial Sequence Primer
cgcctctttgaaagtttctggg

SEQ ID NO: 16 - DNA, 22 bp, Artificial Sequence
  Description of Artificial Sequence Primer
ctcacagtctctggtgagggag

SEQ ID NO: 17 - DNA, 24 bp, Artificial Sequence
  Description of Artificial Sequence Primer
tatcactttgaggtagtgagagac

SEQ ID NO: 18 - DNA, 23 bp, Artificial Sequence
  Description of Artificial Sequence Primer
tacgttataagacaatctatggg

SEQ ID NO: 19 - DNA, 1314 bp, Murine sp.
atgacttcctggcagaggctctgctggcactatcgcctgtggaccctgggttgctacatgctactggccatccttgccctgaaactgtccctcagactgaagtgtgacttcgatgccatggatctggactctgaggaatttcaaagccagtactgcagggatctcctgtacaagaccctgaagctgccagccaagagttccatcaactgctcaggggtcattcgaggggagcagaaagcggtgacccaggctctgctgaataacctggaaattaagaagaagcagcagctcttcacagaggccgactaccttaggatgacagcagactgtgagcacttcaagaccaagaggaagtttatacaggtcccactgagcaaggaagaggccagcttccccattgcgtactccatggtggtgcatgagaagattgagaacttcgaaaggttgctgcgagctgtgtacacccctcagaatgtatactgtgtccacatggatcagaagtcttcagaaccctttaagcaggcagtcagggccatcgtgtcatgcttccccaatgtcttcatagctagtaagttggtgtcagtggtctatgcttcctggtccagggtgcaggctgacctaaactgcatggaagacttgcttcagagccccgtgccatggaaatacctcctgaacacctgtgggacagactttcccatcaaaaccaatgctgagatggtcaaggccctcaagctattgaaagggcagaacagtatggagtcagaggtaccacctccacataaaaaatcccgctggaaatatcactatgaggtgacagacacattgcacatgaccagcaagaggaagacgccgccacctaataacctaaccatgttcactgggaatgcctacatggtggcttctcgagacttcattgaacacgtgttcagtaactcaaaagcccggcaactgatcgagtgggtaaaagacacctatagtcccgatgagcacctttgggccaccctccagcgtgcctcgtggatgcctgggtcagatcccttgcatcgaaaatttgacctgtcagacatgagagccattgcgagactaaccaagtggtacgaccatgagggagacattgagaacggggcaccttacacgtcttgctcaggaatccaccagcgggctgtctgtgtttatgggtcaggggacctgcactggatacttcagaaccatcacctcttggccaacaagtttgacccaaaggtggatgataatgttcttcagtgtttagaagaatatttacgtcacaaagccatctatgggactgaactatga

SEQ ID NO: 20 - protein, 437 aa, Murine sp.
MTSWQRLCWHYRLWTLGCYMLLAILALKLSLRLKCDFDAMDLDSEEFQSQYCRDLLYKTLKLPAKSSINCSGVIRGEQKAVTQALLNNLEIKKKQQLFTEADYLRMTADCEHFKTKRKFIQVPLSKEEASFPIAYSMVVHEKIENFERLLRAVYTPQNVYCVHMDQKSSEPFKQAVRAIVSCFPNVFIASKLVSVVYASWSRVQADLNCMEDLLQSPVPWKYLLNTCGTDFPIKTNAEMVKALKLLKGQNSMESEVPPPHKKSRWKYHYEVTDTLHMTSKRKTPPPNNLTMFTGNAYMVASRDFIEHVFSNSKARQLIEWVKDTYSPDEHLWATLQRASWMPGSDPLHRKFDLSDMRAIARLTKWYDHEGDIENGAPYTSCSGIHQRAVCVYGSGDLHWILQNHHLLANKFDPKVDDNVLQCLEEYLRHKAIYGTEL

(19) United States
(12) Patent Application Publication
(10) Pub. No.: US 2007/0077250 A1
(43) Pub. Date: Apr. 5, 2007

(54) Novel core 2 beta-1,6-N-acetylglycosaminyltransferase gene

(76) Inventors: Bozena Korczak, Toronto (CA); April Lew, Toronto (CA)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/515,764

(22) Filed: Sep. 6, 2006

Related U.S. Application Data
(63) Continuation of application No. 10/445,318, filed on May 27, 2003, now abandoned.

(51) Int. Cl. A61K 48/00; C12Q 1/68; C07H 21/04; C12P 21/06; A61K 39/395; C12N 9/10

(52) U.S. Cl. 424/146.1; 435/6; 435/69.1; 435/193; 435/320.1; 435/325; 514/44; 530/388.26; 536/23.2

(57) ABSTRACT

The invention provides novel core 2 β-1,6-N-acetylglycosaminyltransferase nucleic acids, and polypeptides encoded by the nucleic acids. The invention also provides expression vectors, host cells, agonists, antibodies and antagonists, and methods for treating disorders associated with the enzyme.

### CROSS-REFERENCES TO RELATED APPLICATIONS

This application is related to U.S. patent application Ser. No. 09/495,913 filed Feb. 2, 2000, which claims the benefit of Provisional Patent Application No. 60/118,674 filed Feb. 3, 1999, the teachings of which are incorporated herein by reference.

### FIELD OF THE INVENTION

The invention relates to novel core 2 β-1,6-N-acetylglycosaminyltransferase nucleic acid molecules, polypeptides encoded by such nucleic acid molecules, and uses of the nucleic acid molecules and polypeptides.

### BACKGROUND OF THE INVENTION

The enzyme UDP-GlcNAc:Gal[β] 1,3GalNAc-R (GlcNAc to GalNAc) [β]1,6-N-acetylglucosaminyltransferase (i.e. core 2 β-1,6-N-acetylglycosaminyltransferase) converts core 1 (i.e. Gal[β]1,3GalNAc[α]—O) to core 2 structures (i.e. Gal[β]1,3[GlcNAc[β]1,6]GalNAc[α]—O in the O-linked glycan biosynthesis pathway (Williams and Schachter, 1980 J. Biol. Chem 255:11247, 1980 and Schachter H. and Brockhausen, I, In: Allen, H. J. and Kisailus, E. C. (eds) Glycoconjugates. Composition, Structure, and Function. Marcel Dekker, New York, pp 263-332). Core 2 GlcNAc-T activity is important in the extension of O-linked sugars with poly(N-acetyllactosamine) (i.e. repeating Gal [β] 1-4GlcNAc [β] 1-3). These structures have been associated with malignant transformation (Yousefi et al, 1991) and proliferative activation of lymphocytes (Higgins et al, 1991), they affect cellular adhesion (Zhu and Laine, 1985; Laferte and Dennis, 1988), and they may act as ligands for mammalian lectins (Merkle and Cummings, 1988)

Synthesis of branched, complex core 2-based O-linked structures has been found to be controlled by the relative levels of core 2 GlcNAc-T and [α]2,3 sialyl-T (Whitehouse et al, 1997) which compete for the same core 1 acceptor substrate. Therefore, core 2 is a key enzyme in the modulation of cell-cell interactions through glycosylation of target molecules. For example, glycosylation of PSGL-1 modulated by core 2 GlcNAc-T has been found to be a critical step for binding to P-selectin (Kumar et al, 1996; Li et al, 1996).

Expression of Core 2 GlcNAc-T in diabetic heart has also been associated with a stress-response and myocardial hypertrophy (Nishio Y. et al, J. Clin Invest 1995 Oct.; 96(4): 1759-67). Diabetes and hyperglycemia induces core 2 GlcNAc-T gene expression specifically in cardiac myocytes of rats.

GalNAcαR prevents core 2 synthesis by blocking one enzyme earlier in the O-linked pathway, and it reduces invasion and metastasis. A somatic mutation that prevents UDP-Gal transport into the Golgi blocking O- and N-linked extensions including core 2 structures causes a more severe attenuation of metastasis than a block in either pathway alone, suggesting both O-linked core 2 and N-linked branched oligosaccharides contribute to the malignant phenotype. Most recently, it was demonstrated that an increased expression of core 2 GlcNAc-T in colorectal cancer cells is closely correlated with the progression of the disease (Shimodaira K., at al 97, Cancer Res.).

The identification of new core 2 GlcNAc-transferases and nucleic acids encoding the enzymes satisfies a need in the art by providing new compositions which are useful in the diagnosis, prevention, and treatment of disorders mediated by the enzymes including cancer, cardiovascular disorders, and inflammatory disorders.

The citation of any reference herein is not an admission that such reference is available as prior art to the instant invention.

### SUMMARY OF THE INVENTION

The present inventors have identified novel core 2 β-1,6-N-acetylglycosaminyltransferase nucleic acid molecules, and polypeptides encoded by such nucleic acid molecules. The nucleic acid molecules are herein designated “core2b GlcNAc-T” or “core2b GlcNAc-T”, and the polypeptides are herein designated “Core 2b”, “Core 2b GlcNAc-T”, or “Core 2b GlcNAc-T Polypeptide”. The core 2b GlcNAc-T nucleic acid molecules were found to be primarily expressed in the gastrointestinal tract.

Broadly stated the present invention contemplates an isolated Core 2b GlcNAc-T nucleic acid molecule encoding a polypeptide of the invention, including mRNAs, DNAs, cDNAs, genomic DNAs, PNAs, as well as antisense analogs and biologically, diagnostically, prophylactically, clinically or therapeutically useful variants or fragments thereof, and compositions comprising same.

The invention also contemplates an isolated Core 2b GlcNAc-T polypeptide encoded by a nucleic acid molecule of the invention a truncation, an analog, an allelic or species variation thereof, or a homolog of a polypeptide of the invention or a truncation thereof. (Truncations, analogs, allelic or species variations, and homologs are collectively referred to herein as “Core 2b GlcNAc-T Related Polypeptides”). The polypeptide comprises cytosolic, transmembrane, and catalytic regions.

The nucleic acid molecules of the invention permit identification of untranslated nucleic acid sequences or regulatory sequences that specifically promote expression of genes operatively linked to the promoter regions. Identification and use of such promoter sequences are particularly desirable in instances, such as gene transfer or gene therapy, which may specifically require heterologous gene expression in a limited environment. The invention therefore contemplates a nucleic acid molecule comprising a non-coding sequence such as a 5′ and/or 3″ sequence, preferably a non-coding sequence of core2b GlcNAc-T, preferably a sequence as shown in SEQ. ID. NO. 5 or 6.

The nucleic acid molecules which encode for the mature core2b GlcNAc-T polypeptide (may include only the coding sequence for the mature polypeptide (SEQ ID NO. 1, 3, and 19); the coding sequence for the mature polypeptide and additional coding sequences (e.g. leader or secretory sequences, proprotein sequences); the coding sequence for the mature polypeptide (and optionally additional coding sequence) and non-coding sequence, such as introns or non-coding sequence 5′ and/or 3′ of the coding sequence of the mature polypeptide (e.g. SEQ ID NO 5 and 6).

Therefore, the term “nucleic acid molecule encoding a polypeptide” encompasses a nucleic acid molecule which includes only coding sequence for the polypeptide as well as a nucleic acid molecule which includes additional coding and/or non-coding sequences.

The nucleic acid molecules of the invention may be inserted into an appropriate vector, and the vector may contain the necessary elements for the transcription and translation of an inserted coding sequence. Accordingly, vectors may be constructed which comprise a nucleic acid molecule of the invention, and where appropriate one or more transcription and translation elements linked to the nucleic acid molecule.

Vectors are contemplated within the scope of the invention which comprise regulatory sequences of the invention, as well as chimeric gene constructs wherein a regulatory sequence of the invention is operably linked to a heterologous nucleic acid, and a transcription termination signal.

A vector can be used to transform host cells to express a Core 2b GlcNAc-T Polypeptide or Core 2b GlcNAc-T Related Polypeptide, or a heterologous polypeptide (i.e. a polypeptide not naturally expressed in the host cell). Therefore, the invention further provides host cells containing a vector of the invention. The invention also contemplates transgenic non-human mammals whose germ cells and somatic cells contain a vector comprising a nucleic acid molecule of the invention in particular one that encodes an analog of Core 2b GlcNAc-T, or a truncation of Core 2b GlcNAc-T.

The polypeptides of the invention may be obtained as an isolate from natural cell sources, but they are preferably produced by recombinant procedures. In one aspect the invention provides a method for preparing a Core 2b GlcNAc-T Polypeptide, or a Core 2b GlcNAc-T Related Polypeptide utilizing the purified and isolated nucleic acid molecules of the invention. In an embodiment a method for preparing a Core 2b GlcNAc-T Polypeptide, or a Core 2b GlcNAc-T Related Polypeptide is provided comprising:
(a) transferring a vector of the invention comprising a nucleic acid sequence encoding a Core 2b GlcNAc-T Polypeptide, or a Core 2b GlcNAc-T Related Polypeptide, into a host cell;
(b) selecting transformed host cells from untransformed host cells;
(c) culturing a selected transformed host cell under conditions which allow expression of the Core 2b GlcNAc-T Polypeptide, or a Core 2b GlcNAc-T Related Polypeptide; and
(d) isolating the Core 2b GlcNAc-T Polypeptide, or a Core 2b GlcNAc-T Related Polypeptide.

The invention further broadly contemplates a recombinant Core 2b GlcNAc-T Polypeptide, or a Core 2b GlcNAc-T Related Polypeptide obtained using a method of the invention.

A Core 2b GlcNAc-T Polypeptide, or a Core 2b GlcNAc-T Related Polypeptide of the invention may be conjugated with other molecules, such as polypeptides, to prepare fusion polypeptides or chimeric polypeptides. This may be accomplished, for example, by the synthesis of N-terminal or C-terminal fusion polypeptides.

The invention further contemplates antibodies having specificity against an epitope of a Core 2b GlcNAc-T Polypeptide, or a Core 2b GlcNAc-T Related Polypeptide of the invention. A purified antibody which specifically binds to a Core 2b GlcNAc-T Polypeptide, or a Core 2b GlcNAc-T Related Polypeptide of the invention is provided. Antibodies may be labeled with a detectable substance and used to detect polypeptides of the invention in biological samples, tissues, and cells.

The invention also permits the construction of nucleotide probes that are unique to nucleic acid molecules of the invention and/or to polypeptides of the invention. Therefore, the invention also relates to a probe comprising a sequence encoding a polypeptide of the invention, or a portion (i.e. fragment) thereof. The probe may be labeled, for example, with a detectable substance and it may be used to select from a mixture of nucleic acid molecules a nucleic acid molecule of the invention including nucleic acid molecules coding for a polypeptide which displays one or more of the properties of a polypeptide of the invention.

In accordance with an aspect of the invention there is provided a method of, and products for (i.e. kits), diagnosing and monitoring conditions mediated by core 2b GlcNAc-transferases by determining the presence of nucleic acid molecules and polypeptides of the invention

Still further the invention provides a method for evaluating a test compound for its ability to modulate the biological activity of a Core 2b GlcNAc-T Polypeptide, or a Core 2b GlcNAc-T Related Polypeptide of the invention. For example, a substance which inhibits or enhances the catalytic activity of a Core 2b GlcNAc-T Polypeptide, or a Core 2b GlcNAc-T Related Polypeptide may be evaluated. “Modulate” refers to a change or an alteration in the biological activity of a polypeptide of the invention. Modulation may be an increase or a decrease in activity, a change in characteristics, or any other change in the biological, functional, or immunological properties of the polypeptide.

Compounds which modulate the biological activity of a polypeptide of the invention may also be identified using the methods of the invention by comparing the pattern and level of expression of a nucleic acid molecule or polypeptide of the invention in biological samples, tissues and cells, in the presence, and in the absence of the compounds.

In an embodiment of the invention a method is provided for screening a compound for effectiveness as an antagonist of a polypeptide of the invention, comprising the steps of
a) contacting a sample containing said polypeptide with a compound, under conditions wherein antagonist activity of said polypeptide can be detected, and
b) detecting antagonist activity in the sample.

Methods are also contemplated that identify compounds or substances (e.g. polypeptides) which interact with core2b regulatory sequences (e.g. promoter sequences, enhancer sequences, negative modulator sequences).

The nucleic acid molecules, polypeptides, and substances and compounds identified using the methods of the invention, may be used to modulate the biological activity of a Core 2b GlcNAc-T Polypeptide, or a Core 2b GlcNAc-T Related Polypeptide of the invention, and they may be used in the treatment of conditions mediated by core 2b GlcNAc-Transferases such as inflammatory disorders, liver disorders, gastrointestinal disorders, diabetes, and proliferative diseases such as cancer. Accordingly, the nucleic acid molecules, polypeptides, substances and compounds may be formulated into compositions for administration to individuals suffering from one or more of these conditions. Therefore, the present invention also relates to a composition comprising one or more of a polypeptide, nucleic acid molecule, or substance or compound identified using the methods of the invention, and a pharmaceutically acceptable carrier, excipient or diluent. A method for treating or preventing these conditions is also provided comprising administering to a patient in need thereof, a composition of the invention.

The present invention in another aspect provides means necessary for production of gene-based therapies directed at the gastrointestinal tract. These therapeutic agents may take the form of polynucleotides comprising all or a portion of a nucleic acid molecule of the invention comprising a regulatory sequence of core 2b GlcNAc-T placed in appropriate vectors or delivered to target cells in more direct ways.

Having provided a novel Core 2b GlcNAc-T Polypeptide, and nucleic acids [symbol] the invention accordingly further provides methods for preparing [symbol] or more saccharides including sLe^{x} antigens. In specific [symbol] invention relates to a method for preparing an oligosaccharide comprising contacting a reaction mixture comprising an activated GlcNAc, and an acceptor in the presence of a Core 2b GlcNAc-T Polypeptide, or a Core 2b GlcNAc-T Related Polypeptide of the invention.

In accordance with a further aspect of the invention, there are provided processes for utilizing polypeptides or nucleic acid molecules, for in vitro purposes related to scientific research, synthesis of DNA, and manufacture of vectors.

These and other aspects, features, and advantages of the present invention should be apparent to those skilled in the art from the following drawings and detailed description.

### DESCRIPTION OF THE DRAWINGS

The invention will be better understood with reference to the drawings in which:

FIG. 1 shows an alignment of a nucleic acid molecule of the invention (SEQ. ID. NO.1) and human core 2 DNA (SEQ. ID. NO.13);

FIG. 2 shows an alignment of a polypeptide of the invention and human core 2 polypeptide (Accession No. 544360; SEQ. ID. NO.14);

FIG. 3 is a blot showing expression of core2b GlcNAc-T mRNA in human tissues;

FIG. 4 is a blot showing expression of core2b GlcNAc-T mRNA in gastrointestinal tissues;

FIG. 5 is a blot showing expression of core2b GlcNAc-T in normal (N) and tumor (T) tissues; and

FIG. 6 is a blot of an RT PCR analysis of core 2b GlcNAc-T in samples from normal colon and liver and from patients with colon cancer and liver metastasis.

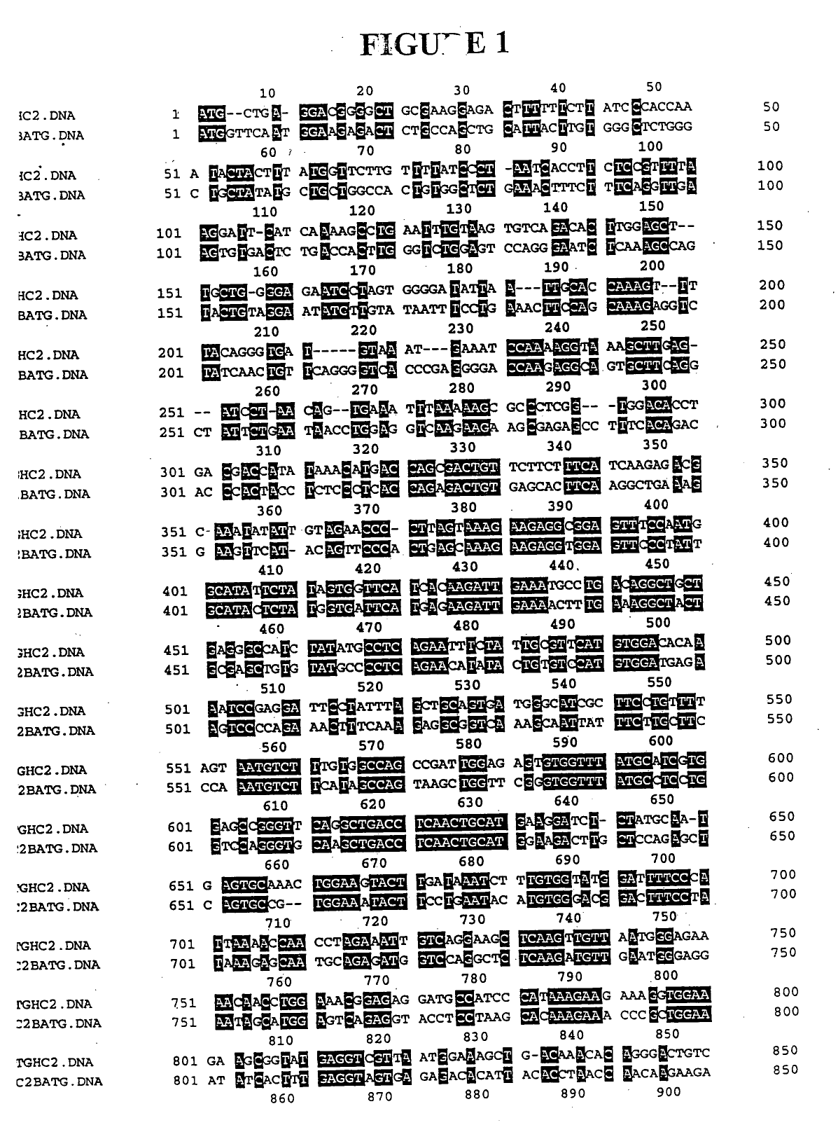

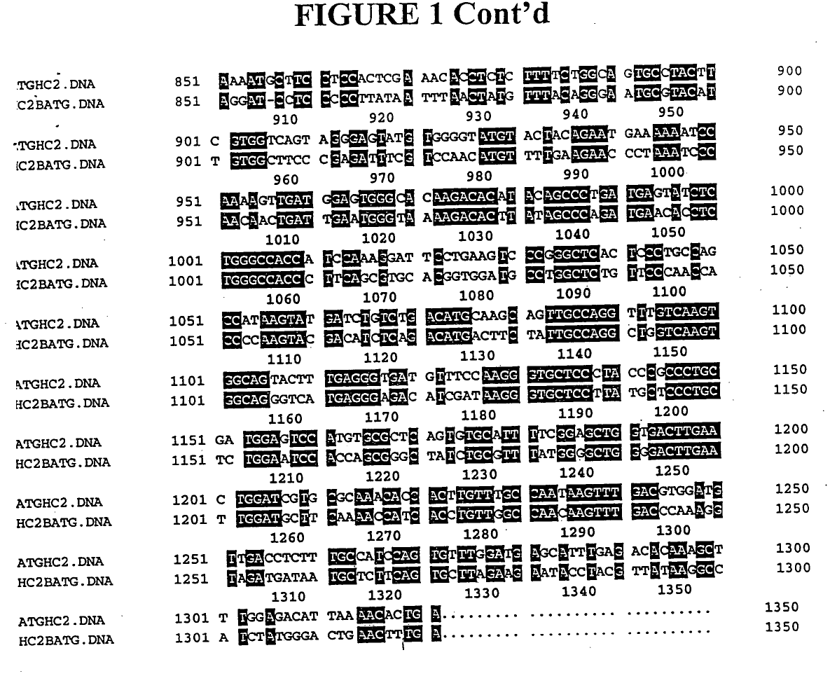

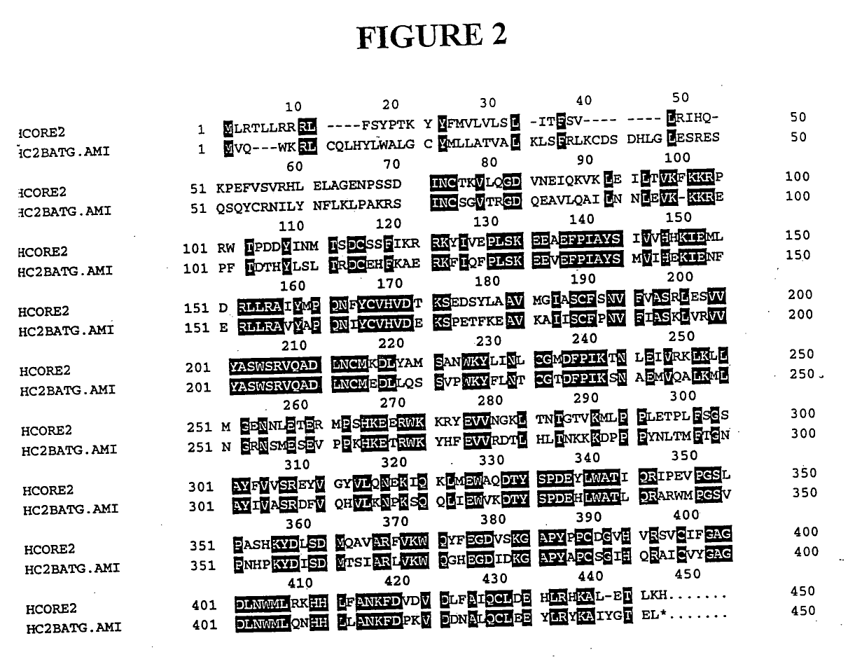

### DETAILED DESCRIPTION OF THE INVENTION

In accordance with the present invention there may be employed conventional molecular biology, microbiology, and recombinant DNA techniques within the skill of the art. Such techniques are explained fully in the literature. See for example, Sambrook, Fritsch, & Maniatis, Molecular Cloning: A Laboratory Manual, Second Edition (1989) Cold Spring Harbor Laboratory Press, Cold Spring Harbor, N.Y.); DNA Cloning: A Practical Approach, Volumes I and II (D. N. Glover ed. 1985); Oligonucleotide Synthesis (M. J. Gait ed. 1984); Nucleic Acid Hybridization B. D. Hames & S. J. Higgins eds. (1985); Transcription and Translation B. D. Hames & S. J. Higgins eds (1984); Animal Cell Culture R. I. Freshney, ed. (1986); Immobilized Cells and enzymes IRL Press, (1986); and B. Perbal, A Practical Guide to Molecular Cloning (1984).

Nucleic Acid Molecules of the Invention

As hereinbefore mentioned, the invention provides isolated Core 2b GlcNAc-T nucleic acid molecules. The term “isolated” refers to a nucleic acid (or polypeptide) removed from its natural environment, purified or separated, or substantially free of cellular material or culture medium when produced by recombinant DNA techniques, or chemical reactants, or other chemicals when chemically synthesized. Preferably, an isolated nucleic acid is at least 60% free, more preferably at least 75% free, and most preferably at least 90 to 99% free from other components with which they are naturally associated. The term “nucleic acid” is intended to include modified or unmodified DNA, RNA, including mRNAs, DNAs, cDNAs, and genomic DNAs, or a mixed polymer, and can be either single-stranded, double-stranded or triple-stranded. For example, a nucleic acid sequence may be a single-stranded or double-stranded DNA, DNA that is a mixture of single- and double-stranded regions, or single-, double- and triple-stranded regions, single- and double-stranded RNA, RNA that may be single-stranded, or more typically, double-stranded, or triple-stranded, or a mixture of regions comprising RNA or DNA, or both RNA and DNA. The strands in such regions may be from the same molecule or from different molecules. The DNAs or RNAs may contain one or more modified bases. For example, the DNAs or RNAs may have backbones modified for stability or for other reasons. A nucleic acid sequence includes an oligonucleotide, nucleotide, or polynucleotide. The term “nucleic acid molecule” and in particular DNA or RNA, refers only to the primary and secondary structure and it does not limit it to any particular tertiary forms.

In an embodiment of the invention an isolated nucleic acid molecule is contemplated which comprises:
(i) a nucleic acid sequence encoding a polypeptide having substantial sequence identity with the amino acid sequence of SEQ. ID. NO. 2, 4, 7, 8, 9, or 20;
(ii) a nucleic acid sequence complementary to (i);
(iii) a nucleic acid sequence differing from any of (i) or (ii) in codon sequences due to the degeneracy of the genetic code;
(iv) a nucleic acid sequence comprising at least 10, 15, 18, preferably at least 20 nucleotides capable of hybridizing to a nucleic acid sequence of SEQ. ID. NO. 1, 3, 10, 11, 12, or 19 or to a degenerate form thereof;
(v) a nucleic acid sequence encoding a truncation, an analog, an allelic or species variation of a polypeptide comprising the amino acid sequence of SEQ. ID. NO. 2, 4, 7, 8, 9, or 20; or
(vi) a fragment, or allelic or species variation of (i), (ii) or (iii)

In a specific embodiment, the isolated nucleic acid molecule comprises:
(i) a nucleic acid sequence having substantial sequence identity or sequence similarity with a nucleic acid sequence shown in SEQ. ID. NO. 1, 3, 10, 11, 12, or 19;
(ii) a nucleic acid sequence complementary to (i), preferably complementary to the full nucleic acid sequence shown in SEQ. ID. NO. 1, 3, 10, 11, 12, or 19;
(iii) a nucleic acid sequence differing from any of the nucleic acid sequences of (i) or (ii) in codon sequences due to the degeneracy of the genetic code; or
(iv) a fragment, or allelic or species variation of (i), (ii) or (iii).

The term “complementary” refers to the natural binding of nucleic acid molecules under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between the single stranded molecules.

In a preferred embodiment the isolated nucleic acid comprises a nucleic acid sequence encoding a polypeptide having an amino acid sequence of SEQ. ID. NO. 2, 4, or 20, or comprises the nucleic acid sequence of SEQ. ID. NO. 1, 3, 10, 11, 12, or 19 wherein T can also be U.

The terms “sequence similarity” or “sequence identity” refer to the relationship between two or more amino acid or nucleic acid sequences, determined by comparing the sequences, which relationship is generally known as “homology”. Identity in the art also means the degree of sequence relatedness between amino acid or nucleic acid sequences, as the case may be, as determined by the match between strings of such sequences. Both identity and similarity can be readily calculated (Computational Molecular Biology, Lesk, A. M., ed., Oxford University Press New York, 1988; Biocomputing: Informatics and Genome Projects, Smith, D. W. ed., Academic Press, New York, 1993; Computer Analysis of Sequence Data, Part I, Griffin, A. M., and Griffin, H. G. eds. Humana Press, New Jersey, 1994; Sequence Analysis in Molecular Biology, von Heinje, G., Academic Press, New York, 1987; and Sequence Analysis Primer, Gribskov, M. and Devereux, J., eds. M. Stockton Press, New York, 1991). While there are a number of existing methods to measure identity and similarity between two amino acid sequences or two nucleic acid sequences, both terms are well 15 known to the skilled artisan (Sequence Analysis in Molecular Biology, von Heinje, G., Academic Press, New York, 1987; Sequence Analysis Primer, Gribskov, M. and Devereux, J., eds. M. Stockton Press, New York, 1991; and Carillo, H., and Lipman, D. SIAM J. Applied Math., 48:1073, 1988). Preferred methods for determining identity are designed to give the largest match between the sequences tested. Methods to determine identity are codified in computer programs. Preferred computer program methods for determining identity and similarity between two sequences include but are not limited to the GCG program package (Devereux, J. et al, Nucleic Acids Research 12(1): 387, 1984), BLASTP, BLASTN, and FASTA (Atschul, S. F. et al., J. Molec. Biol. 215:403, 1990). Identity or similarity may also be determined using the alignment algorithm of Dayhoff et al [Methods in Enzymology 91: 524-545 (1983)].

Preferably, the nucleic acids of the present invention have substantial sequence identity using the preferred computer programs cited herein, for example greater than 50%, 60%, 70%, 75%, 80%, 85%, or 90% identity; more preferably at least 95%, 96% 97%, 98%, or 99% sequence identity to the sequence shown in SEQ. ID. NO. 1, 3, 10, 11, 12, or 19.

Isolated nucleic acids encoding a Core 2b GlcNAc-T Polypeptide and comprising a sequence that differs from the nucleic acid sequence of SEQ. ID. NO. 1, 3, 10, 11, 12, or 19 due to degeneracy in the genetic code are also within the scope of the invention. Such nucleic acids encode equivalent polypeptides but differ in sequence from the sequence of SEQ. ID. NO. 1, 3, 10, 11, 12, or 19 due to degeneracy in the genetic code. As one example, DNA sequence polymorphisms within core2b GlcNAc-T may result in silent mutations that do not affect the amino acid sequence. Variations in one or more nucleotides may exist among individuals within a population due to natural allelic variation. Any and all such nucleic acid variations are within the scope of the invention. DNA sequence polymorphisms may also occur which lead to changes in the amino acid sequence of Core 2b GlcNAc-T Polypeptide. These amino acid polymorphisms are also within the scope of the present invention. In addition, species variations i.e. variations in nucleotide sequence naturally occurring among different species, are within the scope of the invention.

Another aspect of the invention provides a nucleic acid molecule which hybridizes under selective conditions, (e.g. high stringency conditions), to a nucleic acid which comprises a sequence which encodes a Core 2b GlcNAc-T Polypeptide of the invention. Preferably the sequence encodes the amino acid sequence of SEQ. ID. NO. 2, 4, or 20 and comprises at least 10, 15, 18, and preferably at least 20 nucleotides. Selectivity of hybridization occurs with a certain degree of specificity rather than being random. Appropriate stringency conditions which promote DNA hybridization are known to those skilled in the art, or can be found in Current Protocols in Molecular Biology, John Wiley & Sons, N.Y. (1989), 6.3.1-6.3.6. For example, hybridization may occur at 30° C. in 750 mM NaCl, 75 mM trisodium citrate, and 1% SDS, preferably 37° C. in 500 mM NaCl, 500 mM trisodium citrate, 1% SDS, 35% formamide, and 100 g/ml denatured salmon sperm DNA (ssDNA), and more preferably 42° C. in 250 mM NaCl, 25 mM trisodium citrate, 1% SDS, 50% formamide, and 200 μg/ml ssDNA. Useful variations on these conditions will be readily apparent to those skilled in the art.

The stringency may be selected based on the conditions used in the wash step. Wash step stringency conditions may be defined by salt concentration and by temperature. Generally, wash stringency can be increased by decreasing salt concentration or by increasing temperature. By way of example, a stringent salt concentration for the wash step is preferably less than about 30 mM NaCl and 3 mM trisodium citrate, and more preferably less than about 15-mM NaCl and 1.5 mM trisodium citrate. Stringent temperature conditions will generally include temperatures of a least about 25° C., more preferably at least about 68° C. In a preferred embodiment, the wash steps will be carried out at 42° C. in 15 mM NaCl, 1.5 mM trisodium citrate, and 0.1% SDS. In a more preferred embodiment the wash steps are carried out at 68° C. in 15 mM NaCl, 1.5 mM trisodium citrate, and 0.1% SDS. Variations on these conditions will be readily apparent to those skilled in the art.

It will be appreciated that the invention includes nucleic acid molecules encoding a Core 2b GlcNAc-T Polypeptide, or a Core 2b GlcNAc-T Related Polypeptide, including truncations of the polypeptides, allelic and species variants, and analogs of the polypeptides as described herein. In particular, fragments of a nucleic acid of the invention are contemplated that are a stretch of at least about 10, 15, or 18, and preferably at least 20 nucleotides, more typically at least 50 to 200 nucleotides but less than 2 kb. In an embodiment fragments are provided comprising nucleic acid sequences encoding the cytosolic, transmembrane, and catalytic regions of Core 2b GlcNAc-T Polypeptide, or a Core 2b GlcNAc-T Related Polypeptide, (e.g. the sequences shown in SEQ. ID. NO. ID. NO. 10, 11, or 12, respectively). It will further be appreciated that variant forms of the nucleic acid molecules of the invention which arise by alternative splicing of an mRNA corresponding to a cDNA of the invention are encompassed by the invention.

An isolated nucleic acid molecule of the invention which comprises DNA can be isolated by preparing a labeled nucleic acid probe based on all or part of the nucleic acid sequence shown in SEQ. ID. NO. 1 or 3 (e.g. bp 310-766), or 19 (for example SEQ ID NO: 15, 16, 17, 18, or 19) The labeled nucleic acid probe is used to screen an appropriate DNA library (e.g. a cDNA or genomic DNA library). For example, a cDNA library can be used to isolate a cDNA encoding a Core 2b GlcNAc-T Polypeptide, or a Core 2b GlcNAc-T Related Polypeptide by screening the library with the labeled probe using standard techniques. Alternatively, a genomic DNA library can be similarly screened to isolate a genomic clone encompassing a core2 gene. Nucleic acids isolated by screening of a cDNA or genomic DNA library can be sequenced by standard techniques.

An isolated nucleic acid molecule of the invention that is DNA can also be isolated by selectively amplifying a nucleic acid of the invention. “Amplifying” or “amplification” refers to the production of additional copies of a nucleic acid sequence and is generally carried out using polymerase chain reaction (PCR) technologies well known in the art (Dieffenbach, C. W. and G. S. Dveksler (1995) PCR Primer, a Laboratory Manual, Cold Spring Harbor Press, Plainview, N.Y.). In particular, it is possible to design synthetic oligonucleotide primers from the nucleotide sequence shown in SEQ. ID. NO. 1, 3, 10, 11, 12, or 19 for use in PCR. A nucleic acid can be amplified from cDNA or genomic DNA using these oligonucleotide primers and standard PCR amplification techniques. The nucleic acid so amplified can be cloned into an appropriate vector and characterized by DNA sequence analysis. cDNA may be prepared from mRNA, by isolating total cellular mRNA by a variety of techniques, for example, by using the guanidinium-thiocyanate extraction procedure of Chirgwin et al., Biochemistry, 18, 5294-5299 (1979). cDNA is then synthesized from the mRNA using reverse transcriptase (for example, Moloney MLV reverse transcriptase available from Gibco/BRL, Bethesda, Md., or AMV reverse transcriptase available from Seikagaku America, Inc., St. Petersburg, Fla.).

An isolated nucleic acid molecule of the invention which is RNA can be isolated by cloning a cDNA encoding a Core 2b GlcNAc-T Polypeptide, or a Core 2b GlcNAc-T Related Polypeptide into an appropriate vector which allows for transcription of the cDNA to produce an RNA molecule which encodes a Core 2b GlcNAc-T Polypeptide, or a Core 2b GlcNAc-T Related Polypeptide. For example, a cDNA can be cloned downstream of a bacteriophage promoter, (e.g. a T7 promoter) in a vector, cDNA can be transcribed in vitro with T7 polymerase, and the resultant RNA can be isolated by conventional techniques.

A nucleic acid molecule of the invention may be engineered using methods known in the art to alter the core-2b encoding sequence for a variety of purposes including modification of the cloning, processing, and/or expression of the gene product. Procedures such as DNA shuffling by random fragmentation and PCR reassembly of gene fragments and synthetic oligonucleotides may be used to engineer the nucleic acid molecules. Mutations may be introduced by oligonucleotide-mediated site-directed mutagenesis to create for example new restriction sites, alter glycosylation patterns, change codon preference, or produce splice variants.

Nucleic acid molecules of the invention may be chemically synthesized using standard techniques. Methods of chemically synthesizing polydeoxynucleotides are known, including but not limited to solid-phase synthesis which, like peptide synthesis, has been fully automated in commercially available DNA synthesizers (See e.g., Itakura et al. U.S. Pat. No. 4,598,049; Caruthers et al. U.S. Pat. No. 4,458,066; and Itakura U.S. Pat. Nos. 4,401,796 and 4,373,071).

Determination of whether a particular nucleic acid molecule is a core2 or encodes a Core 2b GlcNAc-T Polypeptide, or a Core 2b GlcNAc-T Related Polypeptide can be accomplished by expressing the cDNA in an appropriate host cell by standard techniques, and testing the expressed polypeptide in the methods described herein.

A core 2b GlcNAc-T cDNA or cDNA encoding a Core 2b GlcNAc-T Polypeptide, or a Core 2b GlcNAc-T Related Polypeptide can be sequenced by standard techniques, such as dideoxynucleotide chain termination or Maxam-Gilbert chemical sequencing, to determine the nucleic acid sequence and the predicted amino acid sequence of the encoded polypeptide.

The nucleic acid molecules of the invention may be extended using a partial nucleotide sequence and various PCR-based methods known in the art to detect upstream sequences such as promoters and regulatory elements. For example, restriction-site PCR which uses universal and nested primers to amplify unknown sequences from genomic DNA within a cloning vector may be employed (See Sarkar, G, PCR Methods Applic. 2:318-322, 1993). Inverse PCR which uses primers that extend in divergent directions to amplify unknown sequences from a circularized template may also be used. The template in inverse PCR is derived from restriction fragments adjacent to known sequences in human and yeast artificial chromosome DNA (See e.g. Lagerstrom, M., at al, PCR Methods Applic. 1:111-119, 1991). Other methods for retrieving unknown sequences are known in the art (e.g. Parker, J. D. et al, Nucleic Acids Res. 19:305-306, 1991). In addition, PCR, nested primers, and PROMOTERFINDER libraries (Clontech, Palo Alto, Calif.) may be used to walk genomic DNA. The method is useful in finding intron/exon junctions and avoids the need to screen libraries.

It is preferable when screening for full-length cDNAs to use libraries that have been size-selected to include larger cDNAs. For situations in which an oligo d(T) library does not yield a full-length cDNA, it is preferable to use random-primed libraries which often include sequences containing the 5′ regions of genes. Genomic libraries may be useful for extending the sequence into 5′non-translated regulatory regions.

Commercially available capillary electrophoresis systems may be employed to analyse the size or confirm the sequence of PCR or sequencing products. The system may use flowable polymers for electrophoretic separation, four different nucleotide-specific, laser-stimulated fluorescent dyes, and a charge coupled device camera for detection of the emitted wavelengths. Commercially available software (e.g. GENOTYPER and SEQUENCE NAVIGATOR, Perkin-Elmer) may convert the output/light intensity to electrical signal, and the entire process from loading of samples, and computer analysis and electronic data display may be computer controlled. This procedure may be particularly useful for sequencing small DNA fragments which may be present in limited amounts in a particular sample.

In accordance with one aspect of the invention, a nucleic acid is provided comprising a core 2b GlcNAc-T regulatory sequence such as a promoter sequence. In particular, an isolated nucleic acid is contemplated which comprises:
(i) a nucleic acid sequence having at least 60%, 75-80% identity, 90%, and preferably at least 95% identity with the sequence of SEQ. ID. NO. 5 or 6;
(ii) nucleic acid sequences complementary to (i),
(iii) nucleic acid sequences differing from any of the nucleic acids of (i) or (ii) in codon sequences due to the degeneracy of the genetic code;
(iv) a nucleic acid sequence comprising at least 10, 15, 18, and preferably at least 20 nucleotides, and capable of hybridizing under stringent conditions to a nucleic acid sequence of SEQ. ID. NO. 5 or 6, or to a degenerate form thereof;
(v) a fragment, or allelic or species variation of (i), (ii) or (iii).

In a preferred embodiment, the isolated nucleic acid comprises a nucleic acid sequence of SEQ. ID. NO. 5 or 6, wherein T can also be U.

The invention contemplates nucleic acid molecules comprising all or a portion of a nucleic acid molecule of the invention comprising a regulatory sequence of a core 2b GlcNAc-T (e.g. SEQ ID Nos: 5 or 6) contained in appropriate vectors. The vectors may contain heterologous nucleic acid sequences. “Heterologous nucleic acid” refers to a nucleic acid not naturally located in the cell, or in a chromosomal site of the cell. Preferably, the heterologous nucleic acid includes a nucleic acid foreign to the cell.

In accordance with another aspect of the invention, the nucleic acid molecules isolated using the methods described herein are mutant core2 gene alleles. For example, the mutant alleles may be isolated from individuals either known or proposed to have a genotype which contributes to the symptoms of a condition such as an inflammatory disorder, cancer, or a gastrointestinal disorder. Mutant alleles and mutant allele products may be used in therapeutic and diagnostic methods described herein. For example, a cDNA of a mutant core 2b GlcNAc-T gene may be isolated using PCR as described herein, and the DNA sequence of the mutant allele may be compared to the normal allele to ascertain the mutation(s) responsible for the loss or alteration of function of the mutant gene product. A genomic library can also be constructed using DNA from an individual suspected of or known to carry a mutant allele, or a cDNA library can be constructed using RNA from tissue known, or suspected to express the mutant allele. A nucleic acid encoding a normal core 2b GlcNAc-T gene or any suitable fragment thereof, may then be labeled and used as a probe to identify the corresponding mutant allele in such libraries. Clones containing mutant sequences can be purified and subjected to sequence analysis. In addition, an expression library can be constructed using cDNA from RNA isolated from a tissue of an individual known or suspected to express a mutant core2 allele. Gene products from putatively mutant tissue may be expressed and screened, for example using antibodies specific for a Core 2b GlcNAc-T Polypeptide, or a Core 2b GlcNAc-T Related Polypeptide as described herein. Library clones identified using the antibodies can be purified and subjected to sequence analysis.

Antisense molecules and ribozymes are contemplated within the scope of the invention. They may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding core2b GlcNAc-T. Such DNA sequences may be incorporated into a wide variety of vectors with suitable RNA polymerase promoters such as T7 or SP6. Alternatively, these cDNA constructs that synthesize antisense RNA constitutively or inducibly can be introduced into cell lines, cells, or tissues. RNA molecules may be modified to increase intracellular stability and half-life. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

Polypeptides of the Invention

The polypeptides of the invention are predominately expressed in gastrointestinal tissue (stomach, colon, intestine, testis), and are elevated in cancer (e.g. stomach and liver tumors).

The amino acid sequence of an isolated Core 2b GlcNAc-T Polypeptide of the invention comprises the sequence of SEQ. ID. NO. 2, 4, or 20. In addition to polypeptides comprising the amino acid sequence of SEQ. ID. NO. 2, 4, or 20 the polypeptides of the present invention include truncations, and analogs, allelic and species variations, and homologs of Core 2b GlcNAc-T and truncations thereof as described herein (i.e Core 2b GlcNAc-T Related Polypeptide).

Truncated polypeptides may comprise peptides or fragments having an amino acid sequence of at least five consecutive amino acids in SEQ. ID. NO. 2, 4, or 20 where no amino acid sequence of five or more, six or more, seven or more, or eight or more, consecutive amino acids present in the fragment is present in a polypeptide other than Core 2b GlcNAc-T. In an embodiment of the invention the fragment is a stretch of amino acid residues of at least 12 to 50 contiguous amino acids, preferably 12 to 20 contiguous amino acids, from particular sequences such as the sequences shown in SEQ. ID. NO. 2, 4, or 20. The fragments may be immunogenic and preferably are not immunoreactive with antibodies that are immunoreactive to polypeptides other than Core 2b GlcNAc-T. The fragments may also have core 2b GlcNAc-T activity. In an embodiment the fragments correspond to the cytosolic, transmembrane, or catalytic regions of a Core 2b GlcNAc-T Polypeptide, in particular the fragments shown in SEQ. ID. NO. 7, 8, or 9, respectively.

The truncated polypeptides may have an amino group (—NH2), a hydrophobic group (for example, carbobenzoxyl, dansyl, or T-butyloxycarbonyl), an acetyl group, a 9-fluorenylmethoxy-carbonyl (PMOC) group, or a macromolecule including but not limited to lipid-fatty acid conjugates, polyethylene glycol, or carbohydrates at the amino terminal end. The truncated polypeptides may have a carboxyl group, an amido group, a T-butyloxycarbonyl group, or a macromolecule including but not limited to lipid-fatty acid conjugates, polyethylene glycol, or carbohydrates at the carboxy terminal end.

The polypeptides of the invention may also include analogs of Core 2b GlcNAc-T Polypeptide, and/or truncations thereof as described herein, which may include, but are not limited to Core 2b GlcNAc-T Polypeptide, containing one or more amino acid substitutions, insertions, and/or deletions. Amino acid substitutions may be of a conserved or non-conserved nature. Conserved amino acid substitutions involve replacing one or more amino acids of the Core 2b GlcNAc-T amino acid sequence with amino acids of similar charge, size, and/or hydrophobicity characteristics. When only conserved substitutions are made the resulting analog is preferably functionally equivalent to Core 2b GlcNAc-T. Non-conserved substitutions involve replacing one or more amino acids of the Core 2b GlcNAc-T amino acid sequence with one or more amino acids that possess dissimilar charge, size, and/or hydrophobicity characteristics.

One or more amino acid insertions may be introduced into a Core 2b GlcNAc-T Polypeptide. Amino acid insertions may consist of single amino acid residues or sequential amino acids ranging from about 2 to 15 amino acids in length.

Deletions may consist of the removal of one or more amino acids, or discrete portions from the amino acid sequence. The deleted amino acids may or may not be contiguous. The lower limit length of the resulting analog with a deletion mutation is about 10 amino acids, preferably 100 amino acids.

An allelic variant of Core 2b GlcNAc-T at the polypeptide level differs from one another by only one, or at most, a few amino acid substitutions. A species variation of a Core 2b GlcNAc-T Polypeptide is a variation which is naturally occurring among different species of an organism.

The polypeptides of the invention include homologs of Core 2b Polypeptide and/or truncations thereof as described herein. Such Core 2b GlcNAc-T homologs include polypeptides whose amino acid sequences are comprised of the amino acid sequences of Core 2b Polypeptide regions from other species that hybridize under selective hybridization conditions (see discussion of selective and in particular stringent hybridization conditions herein) with a probe used to obtain a Core 2b GlcNAc-T Polypeptide. These homologs will generally have the same regions which are characteristic of a Core 2b GlcNAc-T Polypeptide. It is anticipated that a polypeptide comprising an amino acid sequence which has at least 58% identity or at least 73% similarity, preferably at least 60-65% identity or at least 80-85% similarity, more preferably at least 70-80% identity or at least 90-95% similarity, most preferably at least 95% identity or at least 99% similarity with the amino acid sequence of SEQ. ID. NO. 2, 4, 7, 8, 9, or 20 will be a homolog of a Core 2 Polypeptide. Polypeptides comprising an amino acid sequence which has at least 19% identify or 42% similarity, preferably at least 30-40% identity or at least 50% similarity, more preferably at least 50-60% identity or at least 60-70% similarity, and most preferably 70-80% identity or at least 80-95% similarity with the amino acid sequence of SEQ. ID. NO. 2, 4, 7, 8, 9, or 20 are also anticipated to be homologs. A percent amino acid sequence similarity or identity is calculated using the methods described herein, preferably the computer programs described herein.

The invention also contemplates isoforms of the polypeptides of the invention. An isoform contains the same number and kinds of amino acids as the polypeptide of the invention, but the isoform has a different molecular structure. The isoforms contemplated by the present invention preferably have the same properties as the polypeptide of the invention as described herein.

The present invention also includes Core 2b GlcNAc-T Polypeptide, or a Core 2b GlcNAc-T Related Polypeptide conjugated with a selected polypeptide, or a marker polypeptide (see below), or other glycosyltransferases to produce fusion polypeptides or chimeric polypeptides.

A Core 2b GlcNAc-T Polypeptide, or a Core 2b GlcNAc-T Related Polypeptide of the invention may be prepared using recombinant DNA methods. Accordingly, the nucleic acids of the present invention having a sequence which encodes a Core 2b GlcNAc-T Polypeptide, or a Core 2b Related Polypeptide of the invention may be incorporated in a known manner into an appropriate vector which ensures good expression of the polypeptide. Possible expression vectors include but are not limited to cosmids, plasmids, phages, or modified viruses (e.g. replication defective retroviruses, adenoviruses and adeno-associated viruses), so long as the vector is compatible with the host cell used.

The invention therefore contemplates a vector of the invention containing a nucleic acid molecule of the invention, and the necessary regulatory sequences for the transcription and translation of the inserted polypeptide-sequence. Suitable regulatory sequences may be derived from a variety of sources, including bacterial, fungal, viral, mammalian, or insect genes (For example, see the regulatory sequences described in Goeddel, Gene Expression Technology: Methods, in Enzymology 185, Academic Press, San Diego, Calif. (1990). Selection of appropriate regulatory sequences is dependent on the host cell chosen as discussed below, and may be readily accomplished by one of ordinary skill in the art. The necessary regulatory sequences may be supplied by the native Core 2b GlcNAc-T Polypeptide and/or its flanking regions.

The invention further provides a vector comprising a nucleic acid of the invention cloned into the expression vector in an antisense orientation. That is, the DNA molecule is linked to a regulatory sequence in a manner which allows for expression, by transcription of the DNA molecule, of an RNA molecule which is antisense to the nucleic acid sequence of SEQ. ID. NO. 1, 3, 5, 6, 10, 11, 12, 15, 16, 17, 18, or 19. Regulatory sequences linked to the antisense nucleic acid can be chosen which direct the continuous expression of the antisense RNA molecule in a variety of cell types, for instance a viral promoter and/or enhancer, or regulatory sequences can be chosen which direct tissue or cell type specific expression of antisense RNA.

The vectors of the invention may also contain a marker gene which facilitates the selection of host cells transformed or transfected with a recombinant molecule of the invention. Examples of marker genes are genes encoding a polypeptide such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. The markers can be introduced on a separate vector from the nucleic acid of interest.

The vectors may also contain genes that encode a fusion moiety which provides increased expression of the recombinant polypeptide; increased solubility of the recombinant polypeptide; and aid in the purification of the target recombinant polypeptide by acting as a ligand in affinity purification. For example, a proteolytic cleavage site may be added to the target recombinant polypeptide to allow separation of the recombinant polypeptide from the fusion moiety subsequent to purification of the fusion polypeptide. Typical fusion expression vectors include pGEX (Amrad Corp., Melbourne, Australia), pMAL (New England Biolabs, Beverly, Mass.) and pRIT5 (Pharmacia, Piscataway, N.J.) which fuse glutathione S-transferase (GST), maltose E binding polypeptide, or polypeptide A, respectively, to the recombinant polypeptide.

The vectors may be introduced into host cells to produce a transformed or transfected host cell. The terms “transfected” and “transfection” encompass the introduction of nucleic acid (e.g. a vector) into a cell by one of many standard techniques. A cell is “transformed” by a nucleic acid when the transfected nucleic acid effects a phenotypic change. Prokaryotic cells can be transfected or transformed with nucleic acid by, for example, electroporation or calcium-chloride mediated transformation. Nucleic acid can be introduced into mammalian cells via conventional techniques such as calcium phosphate or calcium chloride co-precipitation, DEAE-dextran-mediated transfection, lipofectin, electroporation or microinjection. Suitable methods for transforming and transfecting host cells can be found in Sambrook et al. (Molecular Cloning: A Laboratory Manual, 2nd Edition, Cold Spring Harbor Laboratory press (1989)), and other laboratory textbooks.

Suitable host cells include a wide variety of prokaryotic and eukaryotic host cells. For example, the polypeptides of the invention may be expressed in bacterial cells such as E. coli, insect cells (using baculovirus), yeast cells or mammalian cells. Other suitable host cells can be found in Goeddel, Gene Expression Technology: Methods in Enzymology 185, Academic Press, San Diego, Calif. (1991).

A host cell may also be chosen which modulates the expression of an inserted nucleic acid sequence, or modifies (e.g. glycosylation or phosphorylation) and processes (e.g. cleaves) the polypeptide in a desired fashion. Host systems or cell lines may be selected which have specific and characteristic mechanisms for post-translational processing and modification of polypeptides. For example, eukaryotic host cells including CHO, VERO, BHK, A431, HeLA, COS, MDCK, 293, 3T3, and WI38 may be used. For long-term high-yield stable expression of the polypeptide, cell lines and host systems which stably express the gene product may be engineered.

Host cells and in particular cell lines produced using the methods described herein may be particularly useful in screening and evaluating substances and compounds that modulate the activity of a Core 2b GlcNAc-T Polypeptide, or a Core 2b GlcNAc-T Related Polypeptide.

The polypeptides of the invention may also be expressed in non-human transgenic animals including but not limited to mice, rats, rabbits, guinea pigs, micro-pigs, goats, sheep, pigs, non-human primates (e.g. baboons, monkeys, and chimpanzees) (see Hammer et al. (Nature 315:680-683, 1985), Palmiter et al. (Science 222:809-814, 1983), Brinster et al. (Proc Natl Acad. Sci USA 82:44384442, 1985), Palmiter and Brinster (Cell. 41:343-345, 1985) and U.S. Pat. No. 4,736,866). Procedures known in the art may be used to introduce a nucleic acid molecule of the invention encoding a Core 2b GlcNAc-T Polypeptide, or a Core 2b GlcNAc-T Related Polypeptide into animals to produce the founder lines of transgenic animals. Such procedures include pronuclear microinjection, retrovirus mediated gene transfer into germ lines, gene targeting in embryonic stem cells, electroporation of embryos, and sperm-mediated gene transfer.

The present invention contemplates a transgenic animal that carries the core 2b GlcNAc-T gene in all their cells, and animals which carry the transgene in some but not all their cells. The transgene may be integrated as a single transgene or in concatamers. The transgene may be selectively introduced into and activated in specific cell types (See for example, Lasko et al, 1992 Proc. Natl. Acad. Sci. USA 89: 6236). The transgene may be integrated into the chromosomal site of the endogenous gene by gene targeting. The transgene may be selectively introduced into a particular cell type inactivating the endogenous gene in that cell type (See Gu et al Science 265: 103-106).

The expression of a recombinant Core 2b GlcNAc-T Polypeptide, or a Core 2b GlcNAc-T Related Polypeptide in a transgenic animal may be assayed using standard techniques. Initial screening may be conducted by Southern Blot analysis, or PCR methods to analyze whether the transgene has been integrated. The level of mRNA expression in the tissues of transgenic animals may also be assessed using techniques including Northern blot analysis of tissue samples, in situ hybridization, and RT-PCR. Tissues may also be evaluated immunocytochemically using antibodies against a Core 2b GlcNAc-T Polypeptide, or a Core 2b GlcNAc-T Related Polypeptide of the invention.

Polypeptides of the invention may also be prepared by chemical synthesis using techniques well known in the chemistry of polypeptides such as solid phase synthesis (Merrifield, 1964, J. Am. Chem. Assoc. 85:2149-2154) or synthesis in homogenous solution (Houbenweyl, 1987, Methods of Organic Chemistry, ed. E. Wansch, Vol. 15 I and II, Thieme, Stuttgart).

N-terminal or C-terminal fusion polypeptides or chimeric polypeptides comprising a Core 2b GlcNAc-T Polypeptide, or a Core 2b GlcNAc-T Related Polypeptide of the invention conjugated with other molecules, such as polypeptides (e.g. markers or other glycosyltransferases) may be prepared by fusing, through recombinant techniques, the N-terminal or C-terminal of a Core 2b GlcNAc-T Polypeptide, or a Core 2b GlcNAc-T Related Polypeptide, and the sequence of a selected polypeptide or marker polypeptide with a desired biological function. The resultant fusion polypeptides contain a Core 2b GlcNAc-T Polypeptide, or a Core 2b GlcNAc-T Related Polypeptide fused to the selected polypeptide or marker polypeptide as described herein. Examples of polypeptides which may be used to prepare fusion polypeptides include immunoglobulins, glutathione-S-transferase (GST), polypeptide A, hemagglutinin (HA), and truncated myc.

Antibodies

A polypeptide of the invention (including fragments) can be used to prepare antibodies specific for the polypeptides. Antibodies can be prepared which bind a distinct epitope in an unconserved region of the polypeptide. An unconserved region of the polypeptide is one that does not have substantial sequence homology to other polypeptides. A region from a conserved region such as a well-characterized sequence can also be used to prepare an antibody to a conserved region of a polypeptide of the invention. Antibodies having specificity for a polypeptide of the invention may also be raised from fusion polypeptides created by expressing fusion polypeptides in host cells as described herein.

The invention can employ intact monoclonal or polyclonal antibodies, and immunologically active fragments (e.g. a Fab or (Fab)₂ fragment), an antibody heavy chain, and antibody light chain, a genetically engineered single chain F_{V} molecule (Ladner et al, U.S. Pat. No. 4,946,778), humanized antibodies, or a chimeric antibody, for example, an antibody which contains the binding specificity of a murine antibody, but in which the remaining portions are of human origin. Antibodies, including monoclonal and polyclonal antibodies, fragments and chimeras, may be prepared using methods known to those skilled in the art.

Applications of the Nucleic Acid Molecules, Polypeptides, and Antibodies of the Invention

The nucleic acid molecules, Core 2b GlcNAc-T Polypeptide, or a Core 2b GlcNAc-T Related Polypeptide, and antibodies of the invention may be used in the prognostic and diagnostic evaluation of conditions associated with altered expression or activity of a polypeptide of the invention or conditions requiring modulation of a nucleic acid or polypeptide of the invention including inflammatory and proliferative disorders, and gastrointestinal disorders, and the identification of subjects with a predisposition to such conditions (See below). Methods for detecting nucleic acid molecules and polypeptides of the invention, can be used to monitor such conditions (e.g. asthma, rheumatoid arthritis, inflammatory bowel disease, arteriosclerosis, septic shock, ARDS, cancer,) by detecting and localizing the polypeptides and nucleic acids. It would also be apparent to one skilled in the art that the methods described herein may be used to study the developmental expression of the polypeptides of the invention and, accordingly, will provide further insight into the role of the polypeptides. The applications of the present invention also include methods for the identification of substances or compounds that modulate the biological activity of a polypeptide of the invention (See below). The substances, compounds, antibodies etc., may be used for the treatment of conditions requiring modulation of polypeptides of the invention. (See below).

Diagnostic Methods

A variety of methods can be employed for the diagnostic and prognostic evaluation of conditions requiring modulation of a nucleic acid or polypeptide of the invention (e.g. inflammatory disorders, gastrointestinal disorders, liver disorders, and cancer), and the identification of subjects with a predisposition to such conditions. Such methods may, for example, utilize nucleic acid molecules of the invention, and fragments thereof, and antibodies directed against polypeptides of the invention, including peptide fragments. In particular, the nucleic acids and antibodies may be used, for example, for: (1) the detection of the presence of core 2b GlcNAc-T mutations, or the detection of either over- or under-expression of core 2b GlcNAc-T mRNA relative to a non-disorder state or the qualitative or quantitative detection of alternatively spliced forms of core 2b GlcNAc-T transcripts which may correlate with certain conditions or susceptibility toward such conditions; or (2) the detection of either an over- or an under-abundance of a polypeptide of the invention relative to a non-disorder state or the presence of a modified (e.g., less than full length) polypeptide of the invention which correlates with a disorder state, or a progression toward a disorder state.

The methods described herein may be performed by utilizing pre-packaged diagnostic kits comprising for example, at least one specific nucleic acid or antibody described herein, which may be conveniently used, e.g., in clinical settings, to screen and diagnose patients and to screen and identify those individuals exhibiting a predisposition to developing a disorder.

Nucleic acid-based detection techniques and peptide detection techniques are described below. The samples that may be analyzed using the methods of the invention include those that are known or suspected to express core 2b G/cNAc-T or contain a polypeptide of the invention. The methods may be performed on biological samples including but not limited to cells, lysates of cells which have been incubated in cell culture, chromosomes isolated from a cell (e.g. a spread of metaphase chromosomes), genomic DNA (in solutions or bound to a solid support such as for Southern analysis), RNA (in solution or bound to a solid support such as for northern analysis), cDNA (in solution or bound to a solid support), an extract from cells or a tissue, and biological fluids such as serum, urine, blood, and CSF. The samples may be derived from a patient or a culture.

Methods for Detecting Nucleic Acid Molecules of the Invention

The nucleic acid molecules of the invention allow those skilled in the art to construct nucleotide probes for use in the detection of nucleic acid sequences of the invention in biological materials. Suitable probes include nucleic acid molecules based on nucleic acid sequences encoding at least 5 sequential amino acids from regions of the Core 2b GlcNAc-T Polypeptide, or a Core 2b GlcNAc-T Related Polypeptide (see SEQ. ID. No. 1, 3, 10, 11, 12, or 19), preferably they comprise 15 to 50 nucleotides, more preferably 15 to 40 nucleotides, most preferably 15-30 nucleotides. A nucleotide probe may be labeled with a detectable substance such as a radioactive label that provides for an adequate signal and has sufficient half-life such as ³²P, ³H, ¹⁴C or the like. Other detectable substances that may be used include antigens that are recognized by a specific labeled antibody, fluorescent compounds, enzymes, antibodies specific for a labeled antigen, and luminescent compounds. An appropriate label may be selected having regard to the rate of hybridization and binding of the probe to the nucleotide to be detected and the amount of nucleotide available for hybridization. Labeled probes may be hybridized to nucleic acids on solid supports such as nitrocellulose filters or nylon membranes as generally described in Sambrook et al, 1989, Molecular Cloning, A Laboratory Manual (2nd ed.). The nucleic acid probes may be used to detect core 2b GlcNAc-T genes, preferably in human cells. The nucleotide probes may also be useful for example in the diagnosis or prognosis of conditions such as inflammatory disorders, gastrointestinal disorders, liver disorders, kidney disorders, and cancer, and in monitoring the progression of these conditions, or monitoring a therapeutic treatment.

The probe may be used in hybridization techniques to detect a core 2b GlcNAc-T gene. The technique generally involves contacting and incubating nucleic acids (e.g. recombinant DNA molecules, cloned genes) obtained from a sample from a patient or other cellular source with a probe of the present invention under conditions favourable for the specific annealing of the probes to complementary sequences in the nucleic acids. After incubation, the non-annealed nucleic acids are removed, and the presence of nucleic acids that have hybridized to the probe if any are detected.

The detection of nucleic acid molecules of the invention may involve the amplification of specific gene sequences using an amplification method (e.g. PCR), followed by the analysis of the amplified molecules using techniques known to those skilled in the art. Suitable primers can be routinely designed by one of skill in the art. For example, primers may be designed using commercially available software, such as OLIGO 4.06 Primer Analysis software (National Biosciences, Plymouth Minn.) or another appropriate program, to be about 22 to 30 nucleotides in length, to have a GC content of about 50% or more, and to anneal to the template at temperatures of about 68° C. to 72° C.

Genomic DNA may be used in hybridization or amplification assays of biological samples to detect abnormalities involving core 2b GlcNAc-T structure, including point mutations, insertions, deletions, and chromosomal rearrangements. For example, direct sequencing, single stranded conformational polymorphism analyses, heteroduplex analysis, denaturing gradient gel electrophoresis, chemical mismatch cleavage, and oligonucleotide hybridization may be utilized.

Genotyping techniques known to one skilled in the art can be used to type polymorphisms that are in close proximity to the mutations in a core 2b GlcNAc-T gene. The polymorphisms may be used to identify individuals in families that are likely to carry mutations. If a polymorphism exhibits linkage disequalibrium with mutations in the core 2b GlcNAc-T gene, it can also be used to screen for individuals in the general population likely to carry mutations. Polymorphisms which may be used include restriction fragment length polymorphisms (RFLPs), single-base polymorphisms, and simple sequence repeat polymorphisms (SSLPs).

A probe or primer of the invention may be used to directly identify RFLPs. A probe or primer of the invention can additionally be used to isolate genomic clones such as YACs, BACs, PACs, cosmids, phage or plasmids. The DNA in the clones can be screened for SSLPs using hybridization or sequencing procedures.

Hybridization and amplification techniques described herein may be used to assay qualitative and quantitative aspects of core 2b GlcNAc-T expression. For example, RNA may be isolated from a cell type or tissue known to express core 2b GlcNAc-T and tested utilizing the hybridization (e.g. standard Northern analyses) or PCR techniques referred to herein. The techniques may be used to detect differences in transcript size that may be due to normal or abnormal alternative splicing. The techniques may be used to detect quantitative differences between levels of full length and/or alternatively splice transcripts detected in normal individuals relative to those individuals exhibiting symptoms of a disease.

The primers and probes may be used in the above described methods in situ i.e directly on tissue sections (fixed and/or frozen) of patient tissue obtained from biopsies or resections.

Oligonucleotides or longer fragments derived from any of the nucleic acid molecules of the invention may be used as targets in a microarray. The microarray can be used to simultaneously monitor the expression levels of large numbers of genes and to identify genetic variants, mutations, and polymorphisms. The information from the microarray may be used to determine gene function, to understand the genetic basis of a disorder, to diagnose a disorder, to determine predisposition to certain conditions, to treat the disorder, and to develop and monitor the activities of therapeutic agents.

The preparation, use, and analysis of microarrays are well known to a person skilled in the art. (See, for example, Brennan, T. M. et al. (1995) U.S. Pat. No. 5,474,796; Schena, et al. (1996) Proc. Natl. Acad. Sci. 93:10614-10619; Baldeschweiler et al. (1995), PCT Application WO95/251116; Shalon, D. et al. (I 995) PCT application WO95/35505; Heller, R. A. et al. (1997) Proc. Natl. Acad. Sci. 94:2150-2155; and Heller, M. J. et al. (199.7) U.S. Pat. No. 5,605,662.)

Methods for Detecting Polypeptides

Antibodies specifically reactive with a Core 2b GlcNAc-T Polypeptide, a Core 2b GlcNAc-T Related Polypeptide, or derivatives, such as enzyme conjugates or labeled derivatives, may be used to detect Core 2b GlcNAc-T Polypeptides or Core 2b GlcNAc-T Related Polypeptides in various biological materials. They may be used as diagnostic or prognostic reagents and they may be used to detect abnormalities in the level of Core 2b GlcNAc-T Polypeptides or Core 2b GlcNAc-T Related Polypeptides, expression, or abnormalities in the structure, and/or temporal, tissue, cellular, or subcellular location of the polypeptides. Antibodies may also be used to screen potentially therapeutic compounds in vitro to determine their effects on a condition such as an inflammatory disorder, cancer, or gastrointestinal disorders. In vitro immunoassays may also be used to assess or monitor the efficacy of particular therapies. The antibodies of the invention may also be used in vitro to determine the level of Core 2b GlcNAc-T Polypeptide or Core 2b GlcNAc-T Related Polypeptide expression in cells genetically engineered to produce a Core 2b GlcNAc-T Polypeptide or Core 2b GlcNAc-T Related Polypeptide.

The antibodies may be used in any known immunoassays that rely on the binding interaction between an antigenic determinant of a polypeptide of the invention, and the antibodies. Examples of such assays are radioimmunoassays, enzyme immunoassays (e.g. ELISA), immunofluorescence, immunoprecipitation, latex agglutination, hemagglutination, and histochemical tests. The antibodies may be used to detect and quantify polypeptides of the invention in a sample in order to determine their role in particular cellular events or pathological states, and to diagnose and treat such pathological states.

In particular, the antibodies of the invention may be used in immuno-histochemical analyses, for example, at the cellular and sub-subcellular level, to detect a polypeptide of the invention, to localise it to particular cells and tissues, and to specific subcellular locations, and to quantitate the level of expression.

Cytochemical techniques known in the art for localizing antigens using light and electron microscopy may be used to detect a polypeptide of the invention. Generally, an antibody of the invention may be labeled with a detectable substance and a polypeptide may be localised in tissues and cells based upon the presence of the detectable substance. Various methods of labeling polypeptides are known in the art and may be used. Examples of detectable substances include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), luminescent labels such as luminol; enzymatic labels (e.g., horseradish peroxidase, β-galactosidase, luciferase, alkaline phosphatase, acetylcholinesterase), biotinyl groups (which can be detected by marked avidin e.g., streptavidin containing a fluorescent marker or enzymatic activity that can be detected by optical or calorimetric methods), predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualised by electron microscopy.

The antibody or sample may be immobilized on a carrier or solid support which is capable of immobilizing cells, antibodies, etc. For example, the carrier or support may be nitrocellulose, or glass, polyacrylamides, gabbros, and magnetite. The support material may have any possible configuration including spherical (e.g. bead), cylindrical (e.g. inside surface of a test tube or well, or the external surface of a rod), or flat (e.g. sheet, test strip). Indirect methods may also be employed in which the primary antigen-antibody reaction is amplified by the introduction of a second antibody, having specificity for the antibody reactive against a polypeptide of the invention. By way of example, if the antibody having specificity against a polypeptide of the invention is a rabbit IgG antibody, the second antibody may be goat anti-rabbit gamma-globulin labeled with a detectable substance as described herein.

Where a radioactive label is used as a detectable substance, a polypeptide of the invention may be localized by radioautography. The results of radioautography may be quantitated by determining the density of particles in the radioautographs by various optical methods, or by counting the grains.

A polypeptide of the invention may also be detected by assaying for Core 2b GlcNAc-T activity as described herein. For example, a sample may be reacted with an acceptor substrate and a sugar donor under conditions where a Core 2b GlcNAc-T Polypeptide or Core 2b GlcNAc-T Related Polypeptide is capable of transferring the sugar donor to the acceptor substrate to produce a sugar donor-acceptor substrate complex.

Methods for Identifying or Evaluating Substances/Compounds

The methods described herein are designed to identify substances and compounds that modulate the expression or biological activity of a Core 2b GlcNAc-T Polypeptide or Core 2b GlcNAc-T Related Polypeptide including substances that interfere with, or enhance the expression or activity of a Core 2b GlcNAc-T Polypeptide or Core 2b GlcNAc-T Related Polypeptide.

Substances and compounds identified using the methods of the invention include but are not limited to peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)₂, and Fab expression library fragments, and epitope-binding fragments thereof)], polypeptides, nucleic acids, carbohydrates, a monosaccharide, an oligosaccharide or polysaccharide, and small organic or inorganic molecules. A substance or compound may be an endogenous physiological compound or it may be a natural or synthetic compound.

Substances which modulate a Core 2b GlcNAc-T Polypeptide or Core 2b GlcNAc-T Related Polypeptide can be identified based on their ability to associate with a Core 2b GlcNAc-T Polypeptide or Core 2b GlcNAc-T Related Polypeptide. Therefore, the invention also provides methods for identifying substances that associate with a Core 2b GlcNAc-T Polypeptide or Core 2b GlcNAc-T Related Polypeptide. Substances identified using the methods of the invention may be isolated, cloned and sequenced using conventional techniques. A substance that associates with a polypeptide of the invention may be an agonist or antagonist of the biological or immunological activity of a polypeptide of the invention.

The term “agonist” refers to a molecule that increases the amount of, or prolongs the duration of, the activity of the polypeptide. The term “antagonist” refers to a molecule which decreases the biological or immunological activity of the polypeptide. Agonists and antagonists may include proteins, nucleic acids, carbohydrates, or any other molecules that associate with a polypeptide of the invention.

Substances which can associate with a Core 2b GlcNAc-T Polypeptide or Core 2b GlcNAc-T Related Polypeptide may be identified by reacting a Core 2b GlcNAc-T Polypeptide or Core 2b GlcNAc-T Related Polypeptide with a test substance which potentially associates with a Core 2b GlcNAc-T Polypeptide or Core 2b GlcNAc-T Related Polypeptide, under conditions which permit the association, and removing and/or detecting the associated Core 2b GlcNAc-T Polypeptide or Core 2b GlcNAc-T Related Polypeptide and substance. Substance-polypeptide complexes, free substance, or non-complexed polypeptides may be assayed. Conditions which permit the formation of substance-polypeptide complexes may be selected having regard to factors such as the nature and amounts of the substance and the polypeptide.

The substance-polypeptide complex, free substance or non-complexed polypeptides may be isolated by conventional isolation techniques, for example, salting out, chromatography, electrophoresis, gel filtration, fractionation, absorption, polyacrylamide gel electrophoresis, agglutination, or combinations thereof. To facilitate the assay of the components, antibody against a polypeptide of the invention or the substance, or labeled polypeptide, or a labeled substance may be utilized. The antibodies, polypeptides, or substances may be labeled with a detectable substance as described above.

A Core 2b GlcNAc-T Polypeptide or Core 2b GlcNAc-T Related Polypeptide, or the substance used in the method of the invention may be insolubilized. For example, a polypeptide, or substance may be bound to a suitable carrier such as agarose, cellulose, dextran, Sephadex, Sepharose, carboxymethyl cellulose polystyrene, filter paper, ion-exchange resin, plastic film, plastic tube, glass beads, polyamine-methyl vinyl-ether-maleic acid copolymer, amino acid copolymer, ethylene-maleic acid copolymer, nylon, silk, etc. The carrier may be in the shape of, for example, a tube, test plate, beads, disc, sphere etc. The insolubilized polypeptide or substance may be prepared by reacting the material with a suitable insoluble carrier using known chemical or physical methods, for example, cyanogen bromide coupling.

The invention also contemplates a method for evaluating a compound for its ability to modulate the biological activity of a polypeptide of the invention, by assaying for an agonist or antagonist (i.e. enhancer or inhibitor) of the association of the polypeptide with a substance which associates with the polypeptide. The basic method for evaluating if a compound is an agonist or antagonist of the association of a polypeptide of the invention and a substance that associates with the polypeptide, is to prepare a reaction mixture containing the polypeptide and the substance under conditions which permit the formation of substance—polypeptide complexes, in the presence of a test compound. The test compound may be initially added to the mixture, or may be added subsequent to the addition of the polypeptide and substance. Control reaction mixtures without the test compound or with a placebo are also prepared. The formation of complexes is detected and the formation of complexes in the control reaction but not in the reaction mixture indicates that the test compound interferes with the interaction of the polypeptide and substance. The reactions may be carried out in the liquid phase or the polypeptide, substance, or test compound may be immobilized as described herein.

It will be understood that the agonists and antagonists i.e. inhibitors and enhancers, that can be assayed using the methods of the invention may act on one or more of the interaction sites on the polypeptide or substance including agonist binding sites, competitive antagonist binding sites, non-competitive antagonist binding sites or allosteric sites.

The invention also makes it possible to screen for antagonists that inhibit the effects of an agonist of the interaction of a polypeptide of the invention with a substance which is capable of associating with the polypeptide. Thus, the invention may be used to assay for a compound that competes for the same interacting site of a polypeptide of the invention.

Substances that modulate a Core 2b GlcNAc-T Polypeptide or Core 2b GlcNAc-T Related Polypeptide of the invention can be identified based on their ability to interfere with or enhance the activity of a Core 2b GlcNAc-T Polypeptide or Core 2b GlcNAc-T Related Polypeptide. Therefore, the invention provides a method for evaluating a compound for its ability to modulate the activity of a Core 2b GlcNAc-T Polypeptide or Core 2b GlcNAc-T Related Polypeptide comprising (a) reacting an acceptor and a sugar donor for a Core 2b GlcNAc-T Polypeptide or Core 2b GlcNAc-T Related Polypeptide in the presence of a test substance; (b) measuring the amount of sugar donor transferred to acceptor, and (c) carrying out steps (a) and (b) in the absence of the test substance to determine if the substance interferes with or enhances transfer of the sugar donor to the acceptor by the Core 2b GlcNAc-T Polypeptide or Core 2b GlcNAc-T Related Polypeptide.

Suitable acceptors for use in the methods of the invention are a saccharide, oligosaccharides, polysaccharides, glycopeptides, glycopolypeptides, or glycolipids which are either synthetic with linkers at the reducing end or naturally occurring structures, for example, asialo-agalacto-fetuin glycopeptide. Acceptors will generally include β-D-galactosyl-1,3-N-acetyl-D-galactosaminyl-.

The sugar donor may be a nucleotide sugar, dolichol-phosphate-sugar or dolichol-pyrophosphate-oligosaccharide, for example, uridine diphospho-N-acetylglucosamine (UDP-GlcNAc), or derivatives or analogs thereof. The Core 2b GlcNAc-T Polypeptide or Core 2b GlcNAc-T Related Polypeptide may be obtained from natural sources or produced used recombinant methods as described herein.

The acceptor or sugar donor may be labeled with a detectable substance as described herein, and the interaction of the polypeptide of the invention with the acceptor and sugar donor will give rise to a detectable change. The detectable change may be colorimetric, photometric, radiometric, potentiometric, etc. The activity of a Core 2b GlcNAc-T Polypeptide or Core 2b GlcNAc-T Related Polypeptide of the invention may also be determined using methods based on HPLC (Koenderman et al., FEBS Lett. 222:42, 1987) or methods employed synthetic oligosaccharide acceptors attached to hydrophobic aglycones (Palcic et al Glycoconjugate 5:49, 1988; and Pierce et al, Biochem. Biophys. Res. Comm. 146: 679, 1987).

The Core 2b GlcNAc-T Polypeptide or Core 2b GlcNAc-T Related Polypeptide is reacted with the acceptor and sugar donor at a pH and temperature and in the presence of a metal cofactor, usually a divalent cation like manganese, effective for the polypeptide to transfer the sugar donor to the acceptor, and where one of the components is labeled, to produce a detectable change. It is preferred to use a buffer with the acceptor and sugar donor to maintain the pH within the pH range effective for the polypeptides. The buffer, acceptor, and sugar donor may be used as an assay composition. Other compounds such as EDTA and detergents may be added to the assay composition.

The reagents suitable for applying the methods of the invention to evaluate compounds that modulate a Core 2b GlcNAc-T Polypeptide or Core 2b GlcNAc-T Related Polypeptide may be packaged into convenient kits providing the necessary materials packaged into suitable containers. The kits may also include suitable supports useful in performing the methods of the invention.

Substances that modulate a Core 2b GlcNAc-T Polypeptide or Core 2b GlcNAc-T Related Polypeptide can also be identified by treating immortalized cells which express the polypeptide with a test substance, and comparing the morphology of the cells with the morphology of the cells in the absence of the substance and/or with immortalized cells which do not express the polypeptide. Examples of immortalized cells that can be used include lung epithelial cell lines such as Mv1Lu or HEK293 (human embryonic kidney) transfected with a vector containing a nucleic acid of the invention. In the absence of an inhibitor the cells show signs of morphologic transformation (e.g. fibroblastic morphology, spindle shape and pile up; the cells are less adhesive to substratum; there is less cell to cell contact in monolayer culture; there is reduced growth-factor requirements for survival and proliferation; the cells grow in soft-agar of other semi-solid medium; there is a lack of contact inhibition and increased apoptosis in low-serum high density cultures; there is enhanced cell motility, and there is invasion into extracellular matrix and secretion of proteases). Substances that inhibit one or more phenotypes may be considered an inhibitor.

A substance that inhibits a Core 2b GlcNAc-T Polypeptide or Core 2b GlcNAc-T Related Polypeptide may be identified by treating a cell which expresses the polypeptide with a test substance, and assaying for complex core 2-based O-linked structures (e.g. repeating Gal[β] 1-4GlcNAc[β]) associated with the cell. The complex core 2-based O-linked structures can be assayed using a substance that binds to the structures (e.g. antibodies). Cells that have not been treated with the substance or which do not express the polypeptide may be employed as controls.

Substances which inhibit transcription or translation of a core 2b gene may be identified by transfecting a cell with an expression vector comprising a recombinant molecule of the invention, including a reporter gene, in the presence of a test substance and comparing the level of expression of the Core 2b GlcNAc-T Polypeptide or Core 2b GlcNAc-T Related Polypeptide, or the expression of the protein encoded by the reporter gene, with a control cell transfected with the nucleic acid molecule in the absence of the substance. The method can be used to identify transciption and translation inhibitors of a core 2b gene.

Compositions and Treatments

The substances or compounds identified by the methods described herein, polypeptides, nucleic acid molecules, and antibodies of the invention may be used for modulating the biological activity of a Core2b GlcNAc-T polypeptide or a Core2b GlcNAc-T Related Polypeptide, and they may be used in the treatment of conditions mediated by Core 2b GlcNAc-Transferases. In particular, they may be used to modulate cellular adhesion associated with a number of disorders including inflammatory disorders, and cancer.

The terms “inflammatory” refers to reactions of both the specific and non-specific defense systems. A specific defense system reaction is a specific immune system reaction to an antigen. Examples of these reactions include antibody response to antigens such as viruses, and delayed-type hypersensitivity. A non-specific defense system reaction is an inflammatory response mediated by leukocytes (including macrophages, eosinophils, and neutrophils) generally incapable of immunological memory. Examples of non-specific reactions include the immediate swelling after a bee sting, and the collection of peripheral mononuclear leukocytes at sites of bacterial infection (pulmonary infiltrates in bacterial pneumonia and pus formation is abscesses).

Treatable disorders include rheumatoid arthritis, post-ischemic leukocyte-mediated tissue damage (reperfusion injury), frost-bite injury or shock, acute leukocyte-mediated lung injury (e.g. adult respiratory distress syndrome (ARDS)), asthma, traumatic shock, septic shock, nephritis, and acute and chronic inflammation including atopic dermatitis, psoriasis, and inflammatory bowel disease. Various platelet-mediated pathologies such as atherosclerosis and clotting can also be treated. The substances and compounds may be useful in minimizing tissue damage accompanying thrombotic disorders. For example, the substances, compounds, antibodies etc. can be of therapeutic value in individuals who have recently experienced stroke, myocardial infarctions, deep vein thrombosis, pulmonary embolism, etc. or in pre-thrombolytic therapy. Inhibitors of Core 2b GlcNAc-T may be useful in reducing angiogenesis as well as leukocyte adhesion and entry into inflamed tissue.

A substance, compound, etc. may be used to treat the secondary effects (e.g. pathological tissue destruction, and/or widespread microcirculatory thrombi and diffuse inflammation) of septic shock or disseminated intravascular coagulation (DIC). Substances compounds, etc. herein may inhibit leukocyte emigration and mitigate tissue damage.

A substance, compound, etc. may also be useful in treating traumatic shock and associated acute tissue injury. Inhibitory substances, compounds etc. may be administered locally or systemically to control tissue damage associated with injuries.

The substances or compounds identified by the methods described herein, antibodies, and polypeptides, and nucleic acid molecules of the invention may be useful in the prevention and treatment of tumors. Tumor metastasis may be inhibited or prevented by inhibiting the adhesion of circulating cancer cells. The substances, compounds, etc. of the invention may be especially useful in the treatment of various forms of neoplasia such as leukemias, lymphomas, melanomas, adenomas, sarcomas, and carcinomas of solid tissues in patients. In particular the composition may be used for treating malignant melanoma, pancreatic cancer, cervico-uterine cancer, cancer of the liver, kidney, stomach, lung, rectum, breast, bowel, gastric, thyroid, neck, cervix, salivary gland, bile duct, pelvis; mediastinum, urethra, bronchogenic, bladder, esophagus and colon, and Kaposi's Sarcoma which is a form of cancer associated with HIV-infected patients with Acquired Immune Deficiency Syndrome (AIDS). The substances etc. are particularly useful in the prevention and treatment of tumors of the gastrointestinal tract including adenomatous polyps (e.g. familial polyposis aid Gardener's syndrome), cancer of the kidney, colon, and small intestine, tumors of the liver, and the metastases derived from these tumors.

Gastrointestinal disorders that may be prevented or treated using the substances or compounds identified by the methods described herein, antibodies, and polypeptides, and nucleic acid molecules of the invention include ascites, cholelithiasis, cholecystitis, cirrhosis, Crohn's disease, diverticulitis, fulminant hepatitis, gastritis, gastric and duodenal ulcers, hepatorenal syndrome, irritable bowel syndrome, jaundice, pancreatitis, and ulcerative colitis.

Other conditions that are treatable with a substance or compound identified in accordance with the methods described herein, antibodies, polypeptides, or nucleic add molecules of the invention are proliferative disorders (e.g. microbial or parasitic infections), kidney disorders, diabetes, and cardiomyopathy. They may also be used to modulate T-cell activation and immunodeficienty due to the Wiskott-Aldrich syndrome or AIDS, or to stimulate hematopoietic progenitor cell growth, and/or confer protection against chemotherapy and radiation therapy in a subject.

Accordingly, the substances, antibodies, and compounds may be formulated into pharmaceutical compositions for administration to subjects in a biologically compatible form suitable for administration in vivo. By “biologically compatible form suitable for administration in vivo” is meant a form of the substance to be administered in which any toxic effects are outweighed by the therapeutic effects. The substances may be administered to living organisms including humans, and animals. Administration of a therapeutically active amount of the pharmaceutical compositions of the present invention is defined as an amount effective, at dosages and for periods of time necessary to achieve the desired result. For example, a therapeutically active amount of a substance may vary according to factors such as the disease state, age, sex, and weight of the individual, and the ability of antibody to elicit a desired response in the individual. Dosage regima may be adjusted to provide the optimum therapeutic response. For example, several divided doses may be administered daily or the dose may be proportionally reduced as indicated by the exigencies of the therapeutic situation.

The active substance may be administered in a convenient manner such as by injection (subcutaneous, intravenous, etc.), oral administration, inhalation, transdermal application, or rectal administration. Depending on the route of administration, the active substance may be coated in a material to protect the compound from the action of enzymes, acids and other natural conditions that may inactivate the compound.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the substances or compounds in association with one or more pharmaceutically acceptable vehicles or diluents, and contained in buffered solutions with a suitable pH and iso-osmotic with the physiological fluids.

After pharmaceutical compositions have been prepared, they can be placed in an appropriate container and labeled for treatment of an indicated condition. For administration of an inhibitor of a polypeptide of the invention, such labeling would include amount, frequency, and method of administration.

The nucleic acid molecules encoding Core2b GlcNAc-T Polypeptides or any fragment thereof, or antisense sequences may be used for therapeutic purposes. Antisense to a nucleic acid molecule encoding a polypeptide of the invention may be used in situations to block the synthesis of the polypeptide. In particular, cells may be transformed with sequences complementary to nucleic acid molecules encoding Core 2b GlcNAc-T Polypeptide. Thus, antisense sequences may be used to modulate Core2b GlcNAc-T activity or to achieve regulation of gene function. Sense or antisense oligomers or larger fragments, can be designed from various locations along the coding or regulatory regions of sequences encoding a polypeptide of the invention.

Expression vectors may be derived from retroviruses, adenoviruses, herpes or vaccinia viruses or from various bacterial plasmids for delivery of nucleic acid sequences to the target organ, tissue, or cells. Vectors that express antisense nucleic acid sequences of core 2b GlcNAc-T can be constructed using techniques well known to those skilled in the art (see for example, Sambrook et al. (supra)).

Genes encoding core2b GlcNAc-T can be turned off by transforming a cell or tissue with expression vectors that express high levels of a nucleic acid molecule or fragment thereof which encodes a polypeptide of the invention. Such constructs may be used to introduce untranslatable sense or antisense sequences into a cell. Even if they do not integrate into the DNA, the vectors may continue to transcribe RNA molecules until all copies are disabled by endogenous nucleases. Transient expression may last for extended periods of time (e.g a month or more) with a non-replicating vector or if appropriate replication elements are part of the vector system.

Modification of gene expression may be achieved by designing antisense molecules, DNA, RNA, or PNA, to the control regions of a core 2b GlcNAc-T gene i.e. the promoters, enhancers, and introns. Preferably the antisense molecules are oligonucleotides derived from the transcription initiation site (e.g. between positions-10 and +10 from the start site). Inhibition can also be achieved by using triple-helix base-pairing techniques. Triple helix pairing causes inhibition of the ability of the double helix to open sufficiently for the binding of polymerases, transcription factors, or regulatory molecules (see Gee J. E. et al (1994) In: Huber, B. E. and B. I. Carr, Molecular and Immunologic Approaches, Futura Publishing Co., Mt. Kisco, N.Y.). An antisense molecule may also be designed to block translation of mRNA by inhibiting binding of the transcript to the ribosomes.

Ribozymes, enzymatic RNA molecules, may be used to catalyze the specific cleavage of RNA. Ribozyme action involves sequence-specific hybridization of the ribozyme molecule to complementary target RNA, followed by endonucleolytic cleavage. For example, hammerhead motif ribozyme molecules may be engineered that can specifically and efficiently catalyze endonucleolytic cleavage of sequences encoding a polypeptide of the invention.

Specific ribosome cleavage sites within any RNA target may be initially identified by scanning the target molecule for ribozyme cleavage sites which include the following sequences: GUA, GUU, and GUC. Short RNA sequences of between 15 and 20 ribonucleotides corresponding to the region of the cleavage site of the target gene may be evaluated for secondary structural features which may render the oligonucleotide inoperable. The suitability of candidate targets may be evaluated by testing accessibility to hybridization with complementary oligonucleotides using ribonuclease protection assays.

Methods for introducing vectors into cells or tissues include those methods discussed herein and which are suitable for in vivo, in vitro and ex vivo therapy. For ex vivo therapy, vectors may be introduced into stem cells obtained from a patient and clonally propagated for autologous transplant into the same patient (See U.S. Pat. Nos. 5,399,493 and 5,437,994). Delivery by transfection and by liposome are well known in the art.

The nucleic acid molecules disclosed herein may also be used in molecular biology techniques that have not yet been developed, provided the new techniques rely on properties of nucleotide sequences that are currently known, including but not limited to such properties as the triplet genetic code and specific base pair interactions.

The invention also provides methods for studying the function of a Core2b GlcNAc-T polypeptide or a Core2b GlcNAc-T Related Polypeptide. Cells, tissues, and non-human animals lacking in core2b GlcNAc-T expression or partially lacking in core2b GlcNAc-T expression may be developed using recombinant expression vectors of the invention having specific deletion or insertion mutations in the core2b GlcNAc-T gene. A recombinant expression vector may be used to inactivate or alter the endogenous gene by homologous recombination, and thereby create a core2b GlcNAc-T deficient cell, tissue, or animal.

Null alleles may be generated in cells, such as embryonic stem cells by deletion mutation. A recombinant core2b GlcNAc-T gene may also be engineered to contain an insertion mutation which inactivates core2b GlcNAc-T. Such a construct may then be introduced into a cell, such as an embryonic stem cell, by a technique such as transfection, electroporation, injection etc. Cells lacking an intact core2b GlcNAc-T gene may then be identified, for example by Southern blotting, Northern Blotting or by assaying for expression of a polypeptide of the invention using the methods described herein. Such cells may then be used to generate transgenic non-human animals deficient in core2b GlcNAc-T. Germline transmission of the mutation may be achieved, for example, by aggregating the embryonic stem cells with early stage embryos, such as 8 cell embryos, in vitro; transferring the resulting blastocysts into recipient females; and, generating germline transmission of the resulting aggregation chimeras. Such a mutant animal may be used to define specific cell populations, developmental patterns and in vivo processes, normally dependent on core2b GlcNAc-T expression.

The invention thus provides a transgenic non-human mammal all of whose germ cells and somatic cells contain a recombinant expression vector that inactivates or alters a gene encoding a Core2b GlcNAc-T polypeptide or a Core2b GlcNAc-T Related Polypeptide. In an embodiment the invention provides a transgenic nonhuman mammal all of whose germ cells and somatic cells contain a recombinant expression vector that inactivates or alters a gene encoding a Core2b GlcNAc-T polypeptide or a Core2b GlcNAc-T Related Polypeptide resulting in a Core2b GlcNAc-T polypeptide or a Core2b GlcNAc-T Related Polypeptide associated pathology. Further the invention provides a transgenic non-human mammal which does not express or has altered expression of a Core2b GlcNAc-T polypeptide or a Core2b GlcNAc-T Related Polypeptide of the invention. In an embodiment, the invention provides a transgenic non-human mammal which does not express or has altered expression of a Core2b GlcNAc-T polypeptide or a Core2b GlcNAc-T Related Polypeptide of the invention resulting in a Core2b GlcNAc-T polypeptide or a Core2b GlcNAc-T Related Polypeptide associated pathology. A Core2b GlcNAc-T polypeptide or a Core2b GlcNAc-T Related Polypeptide pathology refers to a phenotype observed for a Core2b GlcNAc-T polypeptide or a Core2b GlcNAc-T Related Polypeptide homozygous or heterozygous mutant.

A transgenic non-human animal includes but is not limited to mouse, rat, rabbit, sheep, hamster, dog, cat, goat, and monkey, preferably mouse.

The invention also provides a transgenic non-human animal assay system which provides a model system for testing for an agent that reduces or inhibits a pathology associated with a Core2b GlcNAc-T polypeptide or a Core2b GlcNAc-T Related Polypeptide, preferably a Core2b GlcNAc-T polypeptide or a Core2b GlcNAc-T Related Polypeptide associated pathology, comprising:
(a) administering the agent to a transgenic non-human animal of the invention; and
(b) determining whether said agent reduces or inhibits the pathology (e.g. Core2b GlcNAc-T polypeptide or a Core2b GlcNAc-T Related Polypeptide associated pathology) in the transgenic non-human animal relative to a transgenic non-human animal of step (a) which has not been administered the agent.

The agent may be useful in the treatment and prophylaxis of conditions such as inflammatory disorders or cancer as discussed herein. The agents may also be incorporated in a pharmaceutical composition as described herein.

Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The therapeutic index is the dose ratio of therapeutic to toxic effects and it can be expressed as the ED₅₀/LD₅₀ ratio. Pharmaceutical compositions which exhibit large therapeutic indices are preferred.

A polypeptide of the invention may be used to support the survival, growth, migration, and/or differentiation of cells expressing the polypeptide. Thus, a polypeptide of the invention may be used as a supplement to support, for example cells in culture.

Methods for Preparing Oligosaccharides

The invention relates to a method for preparing an oligosaccharide comprising contacting a reaction mixture comprising an activated GlcNAc and an acceptor in the presence of a polypeptide of the invention.

Examples of acceptors for use in the method for preparing an oligosaccharide are a saccharide, oligosaccharides, polysaccharides, glycopeptides, glycopolypeptides, or glycolipids which are either synthetic with linkers at the reducing end or naturally occurring structures, for example, asialo-agalacto-fetuin glycopeptide. The activated GlcNAc may be part of a nucleotide-sugar, a dolichol-phosphate-sugar, or dolichol-pyrophosphate-oligosaccharide.

In an embodiment of the invention, the oligosaccharides are prepared on a carrier that is non-toxic to a mammal, in particular a human, such as a lipid isoprenoid or polyisoprenoid alcohol. An example of a suitable carrier is dolichol phosphate. The oligosaccharide may be attached to a carrier via a labile bond allowing for chemical removal of the oligosaccharide from the lipid carrier. In the alternative, the oligosaccharide transferase may be used to transfer the oligosaccharide from a lipid carrier to a polypeptide.

The following non-limiting examples are illustrative of the present invention:

### EXAMPLE 1

A cDNA sequence of a human Core2b GlcNAc-T homolog (accession number AA397800) was identified by similarity matching using the GeneBank ESTdatabase. This EST cDNA clone was sequenced (937 base pairs) and when translated was shown to be 60% similar (47% identical) to the 3′ end of the human Core2 amino acid sequence. This information initiated a search for the entire sequence of this human Core2-like cDNA using two different methods; screening a human colon cDNA library by colony plaque lifts and 5′ RACE (rapid amplification of cDNA ends).

A human colon cDNA library (Uni-ZAP XR library (Stratagene Cat # 937221) was screened (using standard protocols) with a ³²P-dCTP labeled 485 base pair cDNA probe generated by restriction enzyme digestions of the Core2b GlcNAc-T EST cDNA with Kpn1 and EcoR1. Two and a half million phage clones were screened and 2 positive clones were identified. Each clone was purified to homogeneity by three subsequent rounds of screening. In vivo excision of the pBluescript phagmids of each clone was isolated using conventional methods from Stratagene. The cDNA insert of one clone (clone 6₄₋₄) was excised by EcoR1 and Xho1 digestion and found to be 1350 base pairs in length. This clone was sequenced using T3 and T7 primers and is 73% similar (56% identical) to the 3′ end of human Core2. The cDNA insert of the other clone (clone 30₇) was also sequenced and determined to be 1496 base pairs in length and is a 5′ extension of clone 64-4. Clone 30₇ was designated as hCore2b GlcNAc-T and found to be 75% similar (59% identical) to human Core2. The 5′ RACE protocol was used to isolate the 5′ end of the hCore2b GlcNAc-T cDNA sequence. First strand cDNA synthesis was performed using a PCR primer that was incubated [(primer RACEC2b GlcNAc-T3-3B-CGCCTCTTTGAAAGTTTCTGGG (SEQ. ID. NO. 15)] (100 mMfinal concentration) with 2 μg of mRNA from normal human colon tissue and incubated for 10 minutes at 85° C. and then chilled on ice for 1 minute. To this mixture was added, to final concentrations, 20 mM Tris HCl (pH 8.4), 50 mM KCl, 2.5 mM MgCl₂, 10 mM DTT, 400 μM each dATP, dCTP, dGTP, dTTP and 200 Units of Superscript II RT (GIBCO-BRL) and incubated for 50 minutes at 42° C. The reaction was terminated by placing it at 70° C. for 15 minutes which was then incubated with 2 Units of RNAse and incubated for an additional 30 minutes. The generated cDNA was purified by using GlassMax DNA spin cartridges following the manufacturer's instructions (GIBCO-BRL). The isolated cDNA was tailed with terminal deoxynucleotidyl transferase (TdT) that added homopolymeric dCTP tails to the 3′ ends of the cDNA in a reaction that was incubated for 1.0 minutes at 37° C. with a final composition of 10 mM Tris-HCl (pH 8.4), 25 mM KCl, 1.5 mM MgCl₂, 200 μM dCTP and 1 Unit of TdT. The TdT was heat inactivated for 10 minutes at 65° C. The tailed cDNA (5 μl) was amplified by PCR using two primers [primer RACEC2b GlcNAc-T3-1B-CTCACAGTCTCTGGTGAGGGAG (SEQ. ID. NO. 16)] and an Abridged Anchor primer—sequence not provided from GIBCO-BRL) with the final composition of the reaction as 20 mM Tris-HCl (pH 8.4), 50 mM KCl, 1.5 mM MgCl₂, 400 mM primer RACEC2b GlcNAc-T3-1B, 400 mM Abridged Anchor primer, 200 μM each dATP, dCTP, dGTP, dTTP and 2.5 Units of Taq DNA polymerase. This reaction was transferred to a thermal cycler pre-equilibrated to 94° C. Thirty five cycles of PCR was performed with the following cycling protocol: pre-denaturation at 94° C. for 4 minutes, denaturation at 94° C. for 1 minute, annealing of primers at 62° C. for 2 minutes, primer extension at 72° C. for 2 minutes and final extension at 72° C. for 10 minutes. The 5′ RACE products were analyzed using standard agarose gel electrophoresis protocols. No visible bands were observed, therefore a Southern blot of these products was performed using the identical hCore2b GlcNAc-T probe used for screening. The Southern blot revealed that an approximately 800 bp band specifically hybridized to the probe. Another gel of the 5′ RACE products was run and the region between 1000 and 400 base pairs was isolated using a DNA gel extraction kit from Stratagene and subcloned into the T/A Bluescript vector using standard procedures. Several cDNA fragments were subcloned into the Bluescript vector and were sequenced. One particular clone (537 base pairs) was found to represent the 5′ end of hCore2b GlcNAc-T. The entire Core2b GlcNAc-T cDNA is 438 amino acids in length and is 57% identical and 72% similar to human Core2. The mouse core 2b gene sequence was isolated and sequenced during the preparation of a core 2b knock out mouse. The mouse nucleic acid sequence is SEQ ID NO. 19, and the deduced mouse amino acid sequence is SEQ ID NO. 20.

### EXAMPLE 2

Expression of Core2b GlcNAc-T

Northern Blot Analysis of Human Tissues

Human multiple tissue and tumor cell line Northern blots were obtained from Clontech. The Northern blot containing mRNA from human stomach and liver cancer tissues as well as normal tissues was obtained from Invitrogen. All Northern blots contained 2 μg of mRNA/lane. These blots were hybridized with [α-³²P]dCTP-labeled Kpn1/Egr1 fragment from bp 310 to 766. Amersham multiprime DNA labeling kit and [α-³²P]dCTP (3000 Ci/mol) were used for labeling. Northern blots were hybridized under stringent conditions following the recommended protocol (Clontech) and exposed to x-ray film or phosphoimager.

Results

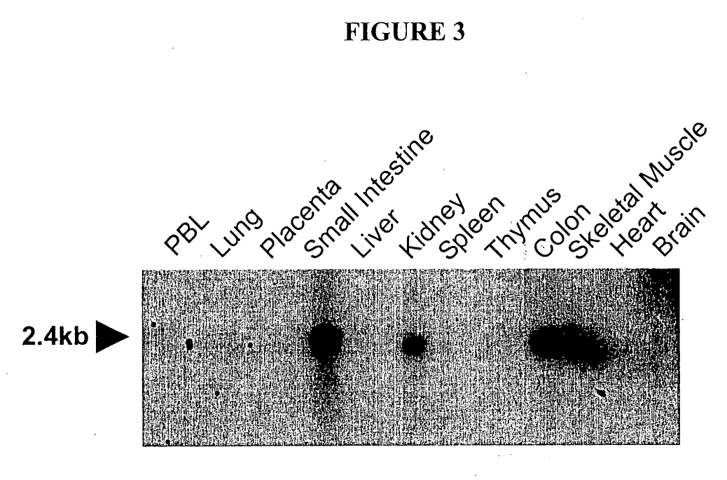

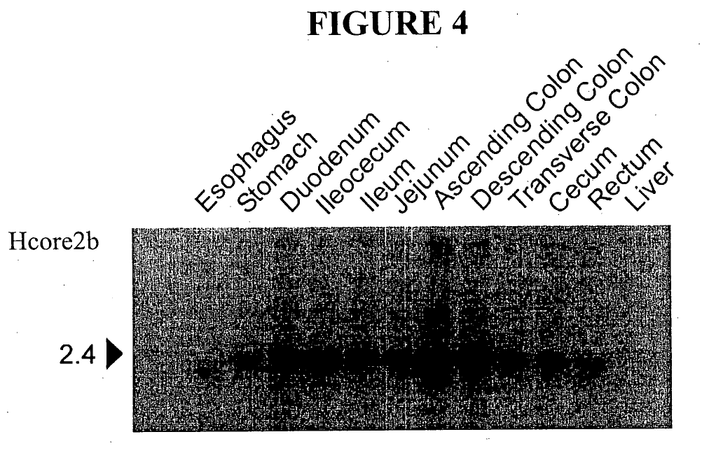

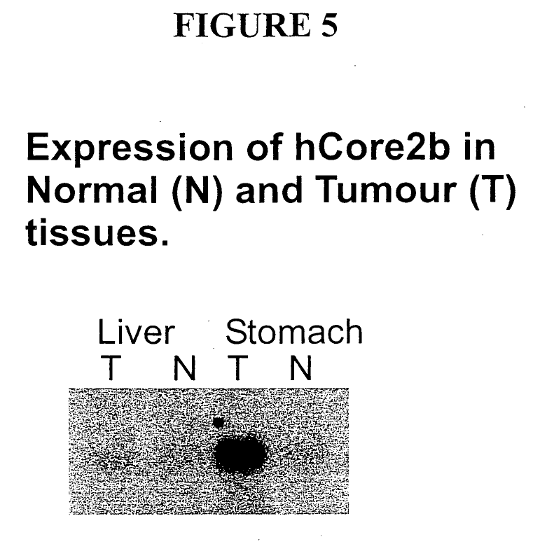

The expression pattern of core2b GlcNAc-T was examined in different human tissues. Hybridization of Core2b GlcNAc-T cDNA probe to Northern blots under stringent conditions revealed the presence of core 2b GlcNAc-T mRNA in colon, kidney, and small intestine (FIG. 3). Further analysis showed wide expression of core 2b GlcNAc-T mRNA throughout human gastrointestinal tissues (FIG. 4). Northern blots from tumor and normal tissues indicated overexpression of core 2b GlcNAc-T mRNA in tumors from liver and stomach when compared to normal tissues (FIG. 5).

### EXAMPLE 3

Quantitative PCR of hcore2b

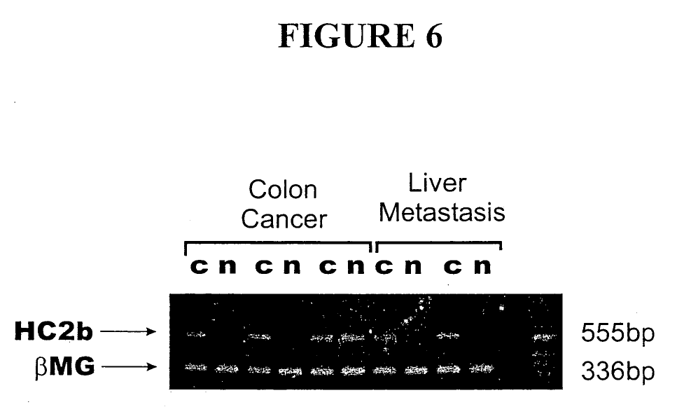

RNA was extracted from each sample (normal and cancerous human colon tissue) using a standard RNA extraction method. Careful quantitation of the extracted RNA was performed. First-strand cDNA synthesis was generated using 5 μg of RNA from each sample, 1 μg of oligo dT (Pharmacia) and H₂O to a volume of 15 μl. Each sample was heated to 70° C. for 5 minutes and then chilled on ice. To 15 μl of this RNA mixture was added 2 μl of 10× PCR buffer (200 mM Tris-HCL (pH 84.), 500 mM KCl), 2 μl of dNTP (10 mM), 0.5 μl of Superscript (GIBCO-BRL) and 0.5 μl of H₂O. The reaction conditions for cDNA synthesis involved 10 minutes at 23° C., 1 hour at 42° C. and 10 minutes at 95° C. Different volumes of each cDNA sample (1 to 7 μl) were tested using a PCR reaction with primers to a housekeeping gene β-Microglobulin (β-MG, Clontech #5438-3). The PCR reaction included the following reagents: 5 μl of 10× PCR buffer (200 mM Tris-HCL (pH 84.), 500 mM KCl), 1.5 μl of 50 mM MgCl, 1 μl of 10 mM dNTP, 1 μl of each of the β-MG primers, cDNA samples (volumes 1-7 μl), 0.5 μl of Platinum Taq polymerase (5 U/μl-GIBCO-BRL) and H₂O to a volume of 50 μl. The reaction conditions included 94° C. for 5 minutes followed by 22 cycles of 94° C. for 1 minute, 60° C. for 1 minute and 72° C. for 1 minute. A linear curve of the β-MG amplified product was observed and each sample was then standardized to an equal amount of β-MG PCR product. Using the standardized cDNA concentrations, hcore2b primers [5′ TATCACTTTGAGGTAGTGAGAGAC3′/5′TACGTTATAAGACAATCTATGGG3′, (SEQ ID. Nos. 17 and 18 respectively)] and the β-MG primers were added to a PCR reaction mixture consisting of 5 μl of 10× PCR buffer (200 mM Tris-HCL (pH 84.), 500 mM KCl), 1.5 μl of 50 mM MgCl, 1 μl of 10 mM dNTP, cDNA samples (volumes 3-6 μl depending on each sample), 0.5 μl of Platinum Taq polymerase (5 U/μl-GIBCO-BRL) and H₂O to a volume of 50 μl. The PCR conditions included 94° C. for 5 minutes followed by 22 cycles of 94° C. for 1 minute, 60° C. for 1 minute and 72° C. for 1 minute. Ten microliters of each PCR sample was run out on an agarose gel for analysis. Core 2b GlcNAc-T mRNA was found in colon cancer and liver metastasis samples (FIG. 6).

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the cell lines, vectors, methodologies etc. which are reported therein which might be used in connection with the invention. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

We claim:

1. An isolated nucleic acid molecule which encodes a core 2b β-1,6-N-acetylglycosaminyltransferase.

2. An isolated nucleic acid molecule as claimed in claim 1 comprising at least 30 nucleotides which hybridizes to SEQ ID NO. 1, 3, or 12 or the complement of SEQ ID NO. 1, 3, or 12 under stringent hybridization conditions.

3. An isolated nucleic acid molecule as claimed in claim 1 which comprises:
(i) a nucleic acid sequence encoding a polypeptide having substantial sequence identity with an amino acid sequence of SEQ. ID. NO. 2, 4, 7, 8, or 9;
(ii) a nucleic acid sequence complementary to (i);
(iii) a nucleic acid sequence differing from any of the nucleic acid sequences of (i) or (ii) in codon sequences due to the degeneracy of the genetic code;
(iv) a nucleic acid sequence comprising at least 10 nucleotides capable of hybridizing under stringent conditions to a nucleic acid sequence of SEQ. ID. NO. 1, 3, or 12 or to a degenerate form thereof;
(v) a nucleic acid sequence encoding a truncation, an analog, an allelic or species variation of a polypeptide comprising an amino acid sequence of SEQ. ID. NO. 2, 4, 7, 8, or 9; or
(vi) a fragment, or allelic or species variation of (i), (ii) or (iii)

4. An isolated nucleic acid molecule as claimed in claim 1 which comprises:
(i) a nucleic acid sequence having substantial sequence identity with a nucleotide sequence of SEQ. ID. NO. 1, 3, 10, 11, or 12;
(ii) a nucleic acid sequence complementary to (i);
(iii) a nucleic acid sequence differing from any of the nucleic acid sequences of (i) to (ii) in codon sequences due to the degeneracy of the genetic code; or
(iv) a fragment, or allelic or species variation of (i), (ii) or (iii).

5. An isolated nucleic acid molecule as claimed in claim 1 which consists essentially of the nucleic acid sequence of SEQ ID NO. 1 or 3.

6. A vector comprising a nucleic acid molecule as claimed in claim 2.

7. A host cell comprising a nucleic acid molecule as claimed in claim 2.

8. An isolated core 2b β-1,6-N-acetylglycosaminyltransferase polypeptide comprising the amino acid sequence of SEQ ID NO. 2, or a naturally occurring variant or fragment thereof having mouse core 2b β-1,6-N-acetylglycosaminyltransferase activity.

9. An isolated core 2b β-1,6-N-acetylglycosaminyltransferase polypeptide comprising an amino acid sequence of SEQ. ID. NO. 2, 4, 7, 8, or 9.

10. An isolated polypeptide as claimed in claim 9 having at least 58% amino acid sequence identity to an amino acid sequence of SEQ. ID. NO. 9.

11. A method for preparing a core 2b β-1,6-N-acetylglycosaminyltransferase comprising:
(a) transferring a vector as claimed in claim 6 into a host cell;
(b) selecting transformed host cells from untransformed host cells;
(c) culturing a selected transformed host cell under conditions which allow expression of the core 2b β-1,6-N-acetylglycosaminyltransferase; and
(d) isolating the core 2b β-1,6-N-acetylglycosaminyltransferase.

12. An antibody having specificity against an epitope of a polypeptide as claimed in claim 9.

13. A probe comprising a sequence encoding a polypeptide as claimed in claim 9, or a part thereof.

14. A method of diagnosing and monitoring conditions mediated by a core 2b α-1,6-N-acetylglycosaminyltransferase by determining the presence of a nucleic acid molecule as claimed in claim 2.

15. A diagnostic or prognostic method for colon cancer or liver metastasis in a subject comprising detecting a nucleic acid molecule as claimed in claim 1 in a sample from the subject.

16. A method of diagnosing and monitoring conditions mediated by a core 2b β-1,6-N-acetylglycosaminyltransferase by determining the presence of a polypeptide as claimed in claim 9.

17. A method for screening a compound for effectiveness as an antagonist of a polypeptide as claimed in claim 9, comprising the steps of
a) contacting a sample containing the protein with a compound, under conditions wherein antagonist activity of the polypeptide can be detected, and
b) detecting antagonist activity in the sample.

18. A compound identified by the method of claim 17.

19. A method for detecting a nucleic acid molecule encoding a polypeptide comprising an amino acid sequence of SEQ. ID. NO. 2, 4, 7, 8, or 9 in a biological sample comprising the steps of:
(a) hybridizing the nucleic acid molecule of claim 3 to nucleic acids of the biological sample, thereby forming a hybridization complex; and
(b) detecting the hybridization complex wherein the presence of the hybridization complex correlates with the presence of a nucleic acid molecule encoding the polypeptide in the biological sample.

20. A method for treating a condition mediated by a core 2b β-1,6-N-acetylglycosaminyltransferase comprising administering an effective amount of an antibody as claimed in claim 12.

21. A gene-based therapy directed at the gastrointestinal tract comprising a polynucleotide comprising all or a portion of a regulatory sequence of SEQ. ID. NO. 5 or 6.
